(19) United States
(12) Patent Application Publication
(10) Pub. No.: US 2023/0338362 A1
(43) Pub. Date: Oct. 26, 2023

(54) METHODS AND COMPOSITIONS FOR TREATING VIRAL INFECTIONS

(71) Applicant: IATERION, INC., Oakland, CA (US)

(72) Inventor: Isaac COHEN, Oakland, CA (US)

(21) Appl. No.: 18/026,567

(22) PCT Filed: Sep. 15, 2021
(86) PCT No.: PCT/US2021/050436

§ 371 Date: Mar. 15, 2023

Related U.S. Application Data
(60) Provisional application No. 63/079,766, filed on Sep. 17, 2020.
(60) Provisional application No. 63/079,794, filed on Sep. 17, 2020.
(60) Provisional application No. 63/079,808, filed on Sep. 17, 2020.
(60) Provisional application No. 63/079,824, filed on Sep. 17, 2020.
(60) Provisional application No. 63/079,837, filed on Sep. 17, 2020.
(60) Provisional application No. 63/079,849, filed on Sep. 17, 2020.
(60) Provisional application No. 63/079,861, filed on Sep. 17, 2020.
(60) Provisional application No. 63/079,866, filed on Sep. 17, 2020.
(60) Provisional application No. 63/079,873, filed on Sep. 17, 2020.
(60) Provisional application No. 63/211,694, filed on Jun. 17, 2021.
(60) Provisional application No. 63/211,697, filed on Jun. 17, 2021.
(60) Provisional application No. 63/211,701, filed on Jun. 17, 2021.
(60) Provisional application No. 63/211,704, filed on Jun. 17, 2021.
(60) Provisional application No. 63/211,711, filed on Jun. 17, 2021.
(60) Provisional application No. 63/211,713, filed on Jun. 17, 2021.
(60) Provisional application No. 63/211,727, filed on Jun. 17, 2021.
(60) Provisional application No. 63/211,729, filed on Jun. 17, 2021.
(60) Provisional application No. 63/211,741, filed on Jun. 17, 2021.

(51) Int. Cl. A61K 31/4741; A61K 31/12; A61K 31/167; A61P 31/16; A61K 45/06; A61K 31/353; A61K 31/7024

(52) U.S. Cl. CPC A61K 31/4741; A61K 31/12; A61K 31/167; A61P 31/16; A61K 45/06; A61K 31/353; A61K 31/7024

(57) ABSTRACT

Antiviral compounds, compositions and method are presented. The composition comprises helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing and an excipient. The excipient may be pharmaceutically acceptable. The excipient may comprise at least one compound that does not occur naturally with helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, or cinnamanilide in nature. The method comprises administering a pharmaceutical composition comprising helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing and a pharmaceutically acceptable excipient to a patient who has, is suspected of having, or is susceptible to a viral infection.

### CROSS REFERENCE TO RELATED APPLICATIONS

This application claims priority under 35 U.S.C. § 119(e) to U.S. Provisional Patent Applications 63/079,766; 63/079,794; 63/079,808; 63/079,824; 63/079,837; 63/079,849; 63/079,861; 63/079,866; and 63/079,873, each of which was filed on Sep. 17, 2020, and each of which is incorporated by reference herein in its entirety. This application also claims priority under 35 U.S.C. § 119(e) to U.S. Provisional Patent Applications 63/211,697; 63/211,704; 63/211,711; 63/211,727; 63/211,741; 63/211,694; 63/211,701; 63/211,713; and 63/211,729, each of which was filed on Jun. 17, 2021, and each of which is incorporated by reference herein in its entirety.

### FIELD

The present invention is directed to antiviral compounds, compositions, and methods.

### BACKGROUND

The current COVID-19 pandemic underscores the continuing need for antiviral therapies to address viral infections caused by known and emergent viruses. SARS-CoV-2, the virus that causes COVID-19, is a novel coronavirus first discovered in human populations in 2019. While preventative measures, such as social distancing, masking, and vaccines may be helpful in attenuating the rate of spread of the virus through a population, many of these measures, and especially vaccination, are considered controversial and their acceptance in the general population has been uneven. Even among the vaccinated population, breakthrough infection is possible, especially for viral variants, such as the especially transmissible delta variant. Other emergent variants are expected and may be inevitable. It is currently impossible to estimate whether, and to what degree, existing vaccines may be effective in preventing or slowing transmission of newly emergent variants of SARS-CoV-2.

Moreover, the origins of SARS-CoV-2 remain to be determined. While the scientific consensus appears to be that the original SARS virus emerged through exposure of miners in Western China to a bat vector, virologists and epidemiologists have yet to agree on the origins of SARS-CoV-2, though various origins have been posited. Definitive proof of its origins may never materialize. Most importantly, whether and when another emergent virus may cause another global pandemic cannot be predicted with any certainty. Given these uncertainties, additional therapeutic options are needed to counter infections of known and emergent viruses, such MERS, SARS, and SARS-CoV-2 and variants and mutants thereof, not to mention perennial influenza and related viruses.

Several antiviral therapeutics have been developed against HIV; and there are a few options for influenza. However, antiviral therapeutic options remain extremely limited for coronaviruses. Recently, the United States Food and Drug Administration (FDA) granted approval of remdesivir for treatment of COVID-19 in hospitalized patients meeting certain other criteria. Despite continued interest in the field, to date, remdesivir is the only antiviral drug approved for treatment of COVID-19. Additional antiviral options are needed for treating coronavirus infections.

In a significant subpopulation of patients infected with SARS-CoV-2, the infection is characterized by severe acute respiratory syndrome. Significant at-risk populations included older and immune compromised patients, as well as those having one or more comorbidities, such as asthma, obesity, diabetes, high blood pressure, atrial fibrillation, and other heart and lung ailments. More recent variants, however, in addition to being more transmissible, also cause severe acute respiratory disease in broader categories of patients, including some who have been previously infected with, or vaccinated against, the original SARS-CoV-2 virus. This has led to intensified interest in additional antiviral strategies.

One key factor may be found in the viral host's immune system, since more lethal forms of the disease are characterized by a runaway immune response or so-called “cytokine storm.” In many viral infections the antiviral cytokine Interferon acts not only to control viral infections, but also to program the adaptive immune response to promote viral clearance. However, in patients with certain preexisting conditions, as well as in patients with severe COVID-19 disease, aberrant interferon and cytokine responses were observed, delaying onset of symptoms, and providing evidence that COVID-19 is an innate immune regulated disease.

Innate immune signaling is the earliest program that alerts host cells to the presence of invading viruses. Pattern Recognition Receptors (PRRs), such as the RIG-1-Like Receptors (RLRs) and Toll-Like Receptors (TLRs), recognize Pathogen Associated Molecular Patterns (PAMPs) from viral components or viral replication intermediates. This recognition results in signaling cascades that initiate an antiviral state in cells. PRRs are distributed on plasma membranes, endosomal membranes, and within the cytosol of host cells to ensure maximal detection of viral PAMPs.

Macrophages are the immune cells in the front line of the body's response to viral infections. There are two kinds of macrophages: those that induce inflammation and those that moderate inflammatory damage. Macrophages neutralize bacteria and viruses using a process called phagocytosis, which engulfs and neutralize the microbes. Macrophages also release chemical signals that trigger an immune response, at the same time, they promote tissue homeostasis and regeneration. The SARS-CoV-2 Spike (S) protein is used for the attachment of the virus to the target cell in the host and thus provides a useful PAMP for in vitro antiviral drug discovery and validation experiments involving one or more elements of the innate immune system.

There is a need for additional and novel antiviral therapeutic compositions for treatment of viral infections. There is also a need for antiviral therapeutic compositions that modulate the innate immune system. The various embodiments disclosed herein address these needs and provide related advantages as well.

### BRIEF SUMMARY OF THE INVENTION

Described herein are antiviral compositions and methods. In some embodiments, there are provided antiviral compositions comprising at least one antiviral compound and at least one additional ingredient. In some embodiments, the antiviral compound is selected from helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, and cinnamanilide, including any antiviral derivative, ester, salt, hydrate, anhydrate, polymorph, or tautomer of any of the foregoing. In some embodiments, there are provided antiviral compositions comprising an antiviral compound, wherein the antiviral compound is helichrysetin, including any antiviral derivative, ester, salt, hydrate, anhydrate, polymorph, or tautomer thereof. In some embodiments, there are provided antiviral compositions comprising an antiviral compound, wherein the antiviral compound is cinanserin, or an antiviral derivative, ester, salt, hydrate, anhydrate, polymorph, or tautomer thereof. In some embodiments, there are provided antiviral compositions comprising an antiviral compound, wherein the antiviral compound is baicalin, or an antiviral derivative, ester, salt, hydrate, anhydrate, polymorph, or tautomer thereof. In some embodiments, there are provided antiviral compositions comprising an antiviral compound, wherein the antiviral compound is fangchinoline, or an antiviral derivative, ester, salt, hydrate, anhydrate, polymorph, or tautomer thereof. In some embodiments, there are provided antiviral compositions comprising an antiviral compound, wherein the antiviral compound is timosaponin B, or an antiviral derivative, ester, salt, hydrate, anhydrate, polymorph, or tautomer thereof. In some embodiments, there are provided antiviral compositions comprising an antiviral compound, wherein the antiviral compound is cepharanthine, or an antiviral derivative, ester, salt, hydrate, anhydrate, polymorph, or tautomer thereof. In some embodiments, there are provided antiviral compositions comprising an antiviral compound, wherein the antiviral compound is tetrandrine, or an antiviral derivative, ester, salt, hydrate, anhydrate, polymorph, or tautomer thereof. In some embodiments, there are provided antiviral compositions comprising an antiviral compound, wherein the antiviral compound is bavachalcone B, or an antiviral derivative, ester, salt, hydrate, anhydrate, polymorph, or tautomer thereof. In some embodiments, there are provided antiviral compositions comprising an antiviral compound, wherein the antiviral compound is rosmarinic acid, or an antiviral derivative, ester, salt, hydrate, anhydrate, polymorph, or tautomer thereof. In some embodiments, there are provided antiviral compositions comprising an antiviral compound, wherein the antiviral compound is formononetin, or an antiviral derivative, ester, salt, hydrate, anhydrate, polymorph, or tautomer thereof. In some embodiments, there are provided antiviral compositions comprising an antiviral compound, wherein the antiviral compound is baicalein, or an antiviral derivative, ester, salt, hydrate, anhydrate, polymorph, or tautomer thereof. In some embodiments, there are provided antiviral compositions comprising an antiviral compound, wherein the antiviral compound is kazinol A, or an antiviral derivative, ester, salt, hydrate, anhydrate, polymorph, or tautomer thereof. In some embodiments, there are provided antiviral compositions comprising an antiviral compound, wherein the antiviral compound is penta-O-beta-glucose hydrate, or an antiviral derivative, ester, salt, hydrate, anhydrate, polymorph, or tautomer thereof. In some embodiments, there are provided antiviral compositions comprising an antiviral compound, wherein the antiviral compound is cinnamanilide, or an antiviral derivative, ester, salt, hydrate, anhydrate, polymorph, or tautomer thereof. In some embodiments, the composition may comprise two or more of the foregoing antiviral compounds, or antiviral derivatives, esters, salts, hydrates, polymorphs, or tautomers thereof. In some embodiments, the compositions may be oral compositions, intranasal compositions, intrapulmonary compositions (e.g., for inhalation), intravenous compositions, subcutaneous compositions, transdermal, sublingual compositions, buccal compositions, intraperitoneal compositions, intrathecal compositions or intracerebroventricular compositions. In some embodiments, the compositions comprise at least one ingredient that does not occur along with the antiviral compound (i.e., helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide) in nature.

Also described herein are antiviral methods, comprising administering to a patient in need thereof an antiviral effective amount of one or more antiviral compounds, as described herein, including antivirally effective derivatives, esters, salts, hydrates, polymorphs or tautomers thereof. In some embodiments, there are provided antiviral methods comprising administration to a patient in need thereof of a composition comprising an antiviral compound selected from the group consisting of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, and cinnamanilide, including any antiviral derivative, ester, salt, hydrate, anhydrate, polymorph, or tautomer of any of the foregoing, and optionally one or more additional ingredients, to a patient in need thereof. In some embodiments, the methods comprise administering a composition comprising helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and optionally one or more pharmaceutically acceptable ingredients orally, intranasally, to the lungs by inhalation, intravenously, transdermally, subcutaneously, sublingually, buccally, or by intraperitoneal or intrathecal injection.

Described herein are compositions having very high in vitro antiviral activity against SARS-CoV2, other coronaviruses (including SARS-CoV, MERS, and common cold viruses) as well as Influenza A and Influenza B, while exhibiting low in vitro toxicity toward uninfected cells. Thus, the methods described herein may be used therapeutically to treat viral infections or prophylactically to reduce the likelihood of developing, or the severity of, a viral infection.

Also described herein are compositions, which have in vitro antiviral and immune modulating activity in the presence of SARS-CoV2, and which are expected to have similar activity in the presence of other coronaviruses (including SARS-CoV, MERS, and common cold viruses) as well as Influenza A and Influenza B. The methods described herein may be used therapeutically to treat viral infections. The methods described herein may be particularly helpful in the treatment of virus-infected patients who are experiencing one or more symptoms of severe viral infection, such as one or more symptoms associated with a cytokine storm.

Other uses and advantages of the various embodiments described herein will be apparent to those skilled in the art upon review of the following disclosure.

### BRIEF DESCRIPTION OF THE FIGURES

The features and advantages of the present invention will become apparent from a consideration of the following detailed description presented in connection with the accompanying drawings in which:

FIG. 1 is a dose response curve for helichrysetin in a SARS-CoV-2 inhibition assay.

FIG. 2 is a toxicity curve for an in vitro VERO-E6 assay of helichrysetin.

FIG. 3 is a dose response curve for cinanserin in a SARS-CoV-2 inhibition assay.

FIG. 4 is a toxicity curve for an in vitro VERO-E6 assay of cinanserin.

FIG. 5 is a dose response curve for baicalin in a SARS-CoV-2 inhibition assay.

FIG. 6 is a toxicity curve for an in vitro VERO-E6 assay of baicalin.

FIG. 7 is a dose response curve for fangchinoline in a SARS-CoV-2 inhibition assay.

FIG. 8 is a toxicity curve for an in vitro VERO-E6 assay of fangchinoline.

FIG. 9 is a dose response curve for timosaponin A in a SARS-CoV-2 inhibition assay.

FIG. 10 is a toxicity curve for an in vitro VERO-E6 assay of timosaponin A.

FIG. 11 is a dose response curve for cepharanthine in a SARS-CoV-2 inhibition assay.

FIG. 12 is a toxicity curve for an in vitro VERO-E6 assay of cepharanthine.

FIG. 13 is a dose response curve for tetrandrine in a SARS-CoV-2 inhibition assay.

FIG. 14 is a toxicity curve for an in vitro VERO-E6 assay of tetrandrine.

FIG. 15 is a dose response curve for bavachalcone B in a SARS-CoV-2 inhibition assay.

FIG. 16 is a toxicity curve for an in vitro VERO-E6 assay of bavachalcone B.

FIG. 17 is a dose response curve for rosmarinic acid in a SARS-CoV-2 inhibition assay.

FIG. 18 is a toxicity curve for an in vitro VERO-E6 assay of rosmarinic acid.

### DETAILED DESCRIPTION OF THE INVENTION

Described herein are antiviral compositions comprising at least one inactive ingredient and at least one antiviral compound, wherein the antiviral compound may be selected from helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, and cinnamanilide, including any antiviral derivative, ester, salt, hydrate, anhydrate, polymorph, or tautomer of any of the foregoing.

Helichrysetin, cinanserin, baicalin, fangchinoline, timosaponin B, cepharanthine, tetrandrine, bavachalcone B, and rosmarinic acid have been shown to have antiviral activity in vivo against SARS-CoV, MERS-CoV, SARS-CoV-2, common cold coronavirus, influenza A and influenza B. Based on in vitro data, it is possible to enter human clinical trials, as taught in the FDA's Guidance for Industry: Antiviral Product Development—Conducting and Submitting Virology Studies to the Agency (June 2006), which is incorporated herein by reference in its entirety, especially pp. 4-5. See also McMahon et al., “New Approaches for Quantitating the Inhibition of HIV-1 Replication by Antiviral Drugs in vitro and in vivo, Curr. Opin. Infect. Dis. 2009 December; 22(6):574-582, which is incorporated herein in its entirety. Thus, as described in more detail herein, there are provided antiviral compositions and methods of treatment, including compositions for antiviral therapy, wherein the compositions comprise one or more inactive ingredients and at least one member of the group consisting of helichrysetin, cinanserin, baicalin, fangchinoline, timosaponin B, cepharanthine, tetrandrine, bavachalcone B, and rosmarinic acid, including any antiviral derivative, salt, hydrate, anhydrate, polymorph, or tautomer of any of the foregoing.

As described herein, fangchinoline is an antiviral compound that selectively induces apoptosis in the SARS-CoV-2 Spike (S) protein treated human macrophages, by activation of caspase 3, within 2 hours of treatment. Fangchinoline also selectively inhibited pro-inflammatory cytokines, such as VEGF, IL-8, GRO, MIP-1β and MMP-9 in SARS-CoV-2 Spike (S) protein treated human macrophages within 2 hours of treatment and induced apoptosis in SARS-CoV-2 Spike (S) protein infected human macrophages through caspase 3 induction. These results indicate that the antiviral compounds enhance type 1 interferon activation, while inhibiting pro-inflammatory cytokine activation and release.

Thus, as described in more detail herein, there are provided antiviral compositions and methods of treatment, including compositions for antiviral therapy, wherein the compositions comprise one or more inactive ingredients and at least one antiviral compound, wherein the antiviral compound is fangchinoline, including any antiviral derivative, salt, hydrate, anhydrate, anhydrate, polymorph, or tautomer thereof. There are also provided immune modulating compositions and methods of treatment, including compositions for immune modulating and antiviral therapy, wherein the compositions comprise one or more inactive ingredients and fangchinoline, including any antiviral derivative, salt, hydrate, anhydrate, anhydrate, polymorph, or tautomer thereof. In some embodiments, the compositions comprise an amount of fangchinoline, or any antiviral derivative, salt, hydrate, anhydrate, polymorph, or tautomer thereof in an amount sufficient to inhibit a pro-inflammatory cytokine, such as VEGF, IL-8, GRO, MIP-1β and MMP-9. In some embodiments, there are provided methods of inhibiting pro-inflammatory cytokine activation and release, enhancing type 1 interferon activation, or both, comprising administering to a patient in need thereof an amount of fangchinoline, or any antiviral derivative, salt, hydrate, anhydrate, polymorph, or tautomer thereof, sufficient to inhibit pro-inflammatory cytokine activation, release, or both. In some embodiments, there are provided antiviral and immune modulating compositions for use in methods of inhibiting pro-inflammatory cytokine activation and release, enhancing type 1 interferon activation, or both, wherein the methods comprise administering to a patient in need thereof an amount of fangchinoline, or any antiviral derivative, salt, hydrate, anhydrate, polymorph, or tautomer thereof, sufficient to inhibit pro-inflammatory cytokine activation, release, or both. In some embodiments, the inhibited cytokines may comprise VEGF, IL-8, GRO, MIP-1β, MMP-9, or combinations of two or more thereof.

As described herein, formononetin is an antiviral compound that selectively induces apoptosis in the SARS-CoV-2 Spike (S) protein treated human macrophages, by activation of caspase 3, within 2 hours of treatment. Formononetin selectively inhibited pro-inflammatory cytokine, such as VEGF, IL-6, IL-8, GRO, MCP-1, MIP-1α, MIP-1β, MMP-9 and RANTES, while inducing anti-inflammatory cytokines, such as IL-10 and IFN-γ, in the SARS-CoV-2 Spike (S) protein treated human macrophages within 2 hours of treatment. These results indicate that the antiviral compounds enhance type 1 interferon activation, while inhibiting pro-inflammatory cytokine activation and release.

Thus, as described in more detail herein, there are provided antiviral compositions and methods of treatment, including compositions for antiviral therapy, wherein the compositions comprise one or more inactive ingredients and at least one antiviral compound, wherein the antiviral compound is formononetin, including any antiviral derivative, salt, hydrate, anhydrate, polymorph, or tautomer thereof. There are also provided immune modulating compositions and methods of treatment, including compositions for immune modulating and antiviral therapy, wherein the compositions comprise one or more inactive ingredients and formononetin, including any antiviral derivative, salt, hydrate, anhydrate, polymorph, or tautomer thereof. In some embodiments, the compositions comprise an amount of formononetin, or any antiviral derivative, salt, hydrate, anhydrate, polymorph, or tautomer thereof, in an amount sufficient to inhibit a pro-inflammatory cytokine, such as VEGF, IL-6, IL-8, GRO, MCP-1, MIP-1α, MIP-1β, MMP-9 and RANTES, while inducing anti-inflammatory cytokines, such as IL-10 and IFN-γ. In some embodiments, there are provided methods of inhibiting pro-inflammatory cytokine activation and release, and/or inducing anti-inflammatory cytokines, such as IL-10 and IFN-γ, comprising administering to a patient in need thereof an amount of formononetin, or any antiviral derivative, salt, hydrate, anhydrate, polymorph, or tautomer thereof, sufficient to inhibit pro-inflammatory cytokine activation, release, or both. In some embodiments, there are provided antiviral and immune modulating compositions for use in methods of inhibiting pro-inflammatory cytokine activation and release, enhancing type 1 interferon activation, or both, inducing anti-inflammatory cytokines, such as IL-10 and IFN-γ, or both, wherein the methods comprise administering to a patient in need thereof an amount of formononetin, or any antiviral derivative, salt, hydrate, anhydrate, polymorph, or tautomer thereof, sufficient to inhibit pro-inflammatory cytokine activation, release, or both, and/or induce anti-inflammatory cytokine activation. In some embodiments, the inhibited cytokines may comprise one or more of VEGF, IL-6, IL-8, GRO, MCP-1, MIP-1α, MIP-1β, MMP-9 and RANTES, and the induced anti-inflammatory cytokines may comprise IL-10 and/or IFN-γ.

As described herein, baicalein is an antiviral compound that selectively induces apoptosis in the SARS-CoV-2 Spike (S) protein treated human macrophages, by activation of caspase 3, within 2 hours of treatment. Baicalein selectively inhibited pro-inflammatory cytokine, such as IL-1α, IL-8, GRO, MCP-1, MIP-1α, MIP-1β, MMP-9 and TNF-α, while inducing anti-inflammatory cytokines such as IL-10, in the SARS-CoV-2 Spike (S) protein treated human macrophages within 2 hours of treatment. These results indicate that the antiviral compounds enhance type 1 interferon activation, while inhibiting pro-inflammatory cytokine activation and release.

Thus, as described in more detail herein, there are provided antiviral compositions and methods of treatment, including compositions for antiviral therapy, wherein the compositions comprise one or more inactive ingredients and at least one antiviral compound, wherein the antiviral compound is baicalein, including any antiviral derivative, salt, hydrate, anhydrate, polymorph, or tautomer thereof. There are also provided immune modulating compositions and methods of treatment, including compositions for immune modulating and antiviral therapy, wherein the compositions comprise one or more inactive ingredients and baicalein, including any antiviral derivative, salt, hydrate, anhydrate, polymorph, or tautomer thereof. In some embodiments, the compositions comprise an amount of baicalein, or any antiviral derivative, salt, hydrate, anhydrate, polymorph, or tautomer thereof, in an amount sufficient to inhibit a pro-inflammatory cytokine, such as IL-1α, IL-8, GRO, MCP-1, MIP-1α, MIP-1β, MMP-9 and TNF-α, while inducing one or more anti-inflammatory cytokines, such as IL-10. In some embodiments, there are provided methods of inhibiting pro-inflammatory cytokine activation and release, and/or inducing anti-inflammatory cytokines, such as IL-10, comprising administering to a patient in need thereof an amount of baicalein, or any antiviral derivative, salt, hydrate, anhydrate, polymorph, or tautomer thereof, sufficient to inhibit pro-inflammatory cytokine activation, release, or both. In some embodiments, there are provided antiviral and immune modulating compositions for use in methods of inhibiting pro-inflammatory cytokine activation and release, enhancing type 1 interferon activation, or both, inducing anti-inflammatory cytokines, such as IL-10, wherein the methods comprise administering to a patient in need thereof an amount of baicalein, or any antiviral derivative, salt, hydrate, anhydrate, polymorph, or tautomer thereof, sufficient to inhibit pro-inflammatory cytokine activation, release, or both, and/or induce anti-inflammatory cytokine activation. In some embodiments, the inhibited cytokines may comprise one or more of VEGF, IL-6, IL-8, GRO, MCP-1, MIP-1α, MIP-1β, MMP-9 and RANTES, and the induced anti-inflammatory cytokine may comprise IL-10.

As described herein, kazinol A is an antiviral compound that selectively induces apoptosis in the SARS-CoV-2 Spike (S) protein treated human macrophages, by activation of caspase 3, within 2 hours of treatment. Kazinol A selectively inhibited pro-inflammatory cytokine, such as IL-1α, IL-4, IL-8, MIP-1β and MMP-9, while inducing anti-inflammatory cytokines such as IL-10, in the SARS-CoV-2 Spike (S) protein treated human macrophages within 2 hours of treatment. These results indicate that kazinol A enhances type 1 interferon activation, while inhibiting pro-inflammatory cytokine activation and release.

Thus, as described in more detail herein, there are provided antiviral compositions and methods of treatment, including compositions for antiviral therapy, wherein the compositions comprise one or more inactive ingredients and at least one antiviral compound, wherein the antiviral compound is kazinol A, including any antiviral derivative, salt, hydrate, anhydrate, polymorph, or tautomer thereof. There are also provided immune modulating compositions and methods of treatment, including compositions for immune modulating and antiviral therapy, wherein the compositions comprise one or more inactive ingredients and kazinol A, including any antiviral derivative, salt, hydrate, anhydrate, polymorph, or tautomer thereof. In some embodiments, the compositions comprise an amount of kazinol A, or any antiviral derivative, salt, hydrate, anhydrate, polymorph, or tautomer thereof, in an amount sufficient to inhibit a pro-inflammatory cytokine, such as IL-1α, IL-4, IL-8, MIP-1β and MMP-9, while inducing one or more anti-inflammatory cytokines, such as IL-10. In some embodiments, there are provided methods of inhibiting pro-inflammatory cytokine activation and release, and/or inducing anti-inflammatory cytokines, such as IL-10, comprising administering to a patient in need thereof an amount of kazinol A, or any antiviral derivative, salt, hydrate, anhydrate, polymorph, or tautomer thereof, sufficient to inhibit pro-inflammatory cytokine activation, release, or both. In some embodiments, there are provided antiviral and immune modulating compositions for use in methods of inhibiting pro-inflammatory cytokine activation and release, enhancing type 1 interferon activation, or both, inducing anti-inflammatory cytokines, such as IL-10, wherein the methods comprise administering to a patient in need thereof an amount of kazinol A, or any antiviral derivative, salt, hydrate, anhydrate, polymorph, or tautomer thereof, sufficient to inhibit pro-inflammatory cytokine activation, release, or both, and/or induce anti-inflammatory cytokine activation. In some embodiments, the inhibited cytokine may comprise one or more of IL-1α, IL-4, IL-8, MIP-1β and MMP-9, and the induced anti-inflammatory cytokine may comprise IL-10.

As described herein, tetrandrine is an antiviral compound that selectively induces apoptosis in the SARS-CoV-2 Spike (S) protein treated human macrophages, by activation of caspase 3, within 2 hours of treatment. Tetrandrine selectively inhibited pro-inflammatory cytokine, such as VEGF, IL-6, IL-8, MIP-1α and MIP-1β, while inducing anti-inflammatory cytokines, such as IL-10, in SARS-CoV-2 Spike (S) protein treated human macrophages within 2 hours of treatment, and induced apoptosis in SARS-CoV-2 Spike (S) protein infected human macrophages through caspase 3 induction.

Thus, as described in more detail herein, there are provided antiviral compositions and methods of treatment, including compositions for antiviral therapy, wherein the compositions comprise one or more inactive ingredients and at least one antiviral compound, wherein the antiviral compound is tetrandrine, including any antiviral derivative, salt, hydrate, anhydrate, polymorph, or tautomer thereof. There are also provided immune modulating compositions and methods of treatment, including compositions for immune modulating and antiviral therapy, wherein the compositions comprise one or more inactive ingredients and tetrandrine, including any antiviral derivative, salt, hydrate, anhydrate, polymorph, or tautomer thereof. In some embodiments, the compositions comprise an amount of tetrandrine, or any antiviral derivative, salt, hydrate, anhydrate, polymorph, or tautomer thereof, in an amount sufficient to inhibit a pro-inflammatory cytokine, such as VEGF, IL-6, IL-8, MIP-1α and MIP-1β, while inducing anti-inflammatory cytokines, such as IL-10. In some embodiments, there are provided methods of inhibiting pro-inflammatory cytokine activation and release, enhancing type 1 interferon activation, or both, and/or inducing anti-inflammatory cytokines, such as IL-10, comprising administering to a patient in need thereof an amount of tetrandrine, or any antiviral derivative, salt, hydrate, anhydrate, polymorph, or tautomer thereof, sufficient to inhibit pro-inflammatory cytokine activation, release, or both. In some embodiments, there are provided antiviral and immune modulating compositions for use in methods of inhibiting pro-inflammatory cytokine activation and release, enhancing type 1 interferon activation, or both, inducing anti-inflammatory cytokines, or both, wherein the methods comprise administering to a patient in need thereof an amount of tetrandrine, or any antiviral derivative, salt, hydrate, anhydrate, polymorph, or tautomer thereof, sufficient to inhibit pro-inflammatory cytokine activation, release, or both, and/or induce anti-inflammatory cytokine activation. In some embodiments, the inhibited cytokine may comprise one or more of VEGF, IL-6, IL-8, MIP-1α and MIP-1β, and the induced anti-inflammatory cytokine may comprise IL-10.

As described herein, cepharanthine is an antiviral compound that selectively induces apoptosis in the SARS-CoV-2 Spike (S) protein treated human macrophages, by activation of caspase 3, within 2 hours of treatment. Cepharanthine selectively inhibited pro-inflammatory cytokine, such as VEGF, IL-6, IL-8, GRO, MIP-1α, MIP-1β and MMP-9 while inducing anti-inflammatory cytokines such as IL-10, in SARS-CoV-2 Spike (S) protein treated human macrophages within 2 hours of treatment. These results indicate that the antiviral compounds enhance type 1 interferon activation, while inhibiting pro-inflammatory cytokine activation and release.

Thus, as described in more detail herein, there are provided antiviral compositions and methods of treatment, including compositions for antiviral therapy, wherein the compositions comprise one or more inactive ingredients and at least one antiviral compound, wherein the antiviral compound is cepharanthine, including any antiviral derivative, salt, hydrate, anhydrate, polymorph, or tautomer thereof. There are also provided immune modulating compositions and methods of treatment, including compositions for immune modulating and antiviral therapy, wherein the compositions comprise one or more inactive ingredients and cepharanthine, including any antiviral derivative, salt, hydrate, anhydrate, polymorph, or tautomer thereof. In some embodiments, the compositions comprise an amount of cepharanthine, or any antiviral derivative, salt, hydrate, anhydrate, polymorph, or tautomer thereof, in an amount sufficient to inhibit a pro-inflammatory cytokine, such as VEGF, IL-6, IL-8, GRO, MIP-1α, MIP-1β and MMP-9, while inducing anti-inflammatory cytokines, such as IL-10. In some embodiments, there are provided methods of inhibiting pro-inflammatory cytokine activation and release, enhancing type 1 interferon activation, or both, and/or inducing anti-inflammatory cytokines, such as IL-10, comprising administering to a patient in need thereof an amount of cepharanthine, or any antiviral derivative, salt, hydrate, anhydrate, polymorph, or tautomer thereof, sufficient to inhibit pro-inflammatory cytokine activation, release, or both. In some embodiments, there are provided antiviral and immune modulating compositions for use in methods of inhibiting pro-inflammatory cytokine activation and release, enhancing type 1 interferon activation, or both, inducing anti-inflammatory cytokines, or both, wherein the methods comprise administering to a patient in need thereof an amount of cepharanthine, or any antiviral derivative, salt, hydrate, anhydrate, polymorph, or tautomer thereof, sufficient to inhibit pro-inflammatory cytokine activation, release, or both, and/or induce anti-inflammatory cytokine activation. In some embodiments, the inhibited cytokine may comprise one or more of VEGF, IL-6, IL-8, GRO, MIP-1α, MIP-1β and MMP-9, and the induced anti-inflammatory cytokine may comprise IL-10.

As described herein, penta-O-galloyl-β-D-glucose hydrate is an immune modulator that selectively induces apoptosis in the SARS-CoV-2 Spike (S) protein treated human macrophages, by activation of caspase 3, within 2 hours of treatment. Penta-O-galloyl-β-D-glucose hydrate selectively inhibited pro-inflammatory cytokine, such as IL-1α, VEGF, IL-1β, IL-2, IL-4, IL-5, IL-6, IL-8, IL-12p70, IL-13, GM-CSF, GRO, MCP-1, MIP-1α, MIP-1β, MMP-9, RANTES, and TNF-α, while inducing anti-inflammatory cytokines such as IL-10, in the SARS-CoV-2 Spike (S) protein treated human macrophages within 2 hours of treatment. These results indicate that the immune modulators enhance type 1 interferon activation, while inhibiting pro-inflammatory cytokine activation and release.

Thus, as described in more detail herein, there are provided antiviral compositions and methods of treatment, including compositions for antiviral therapy, wherein the compositions comprise one or more inactive ingredients and at least one antiviral compound, wherein the antiviral compound is penta-O-galloyl-β-D-glucose hydrate, including any antiviral derivative, salt, hydrate, anhydrate, anhydrate, polymorph, or tautomer thereof. There are also provided immune modulating compositions and methods of treatment, including compositions for immune modulating and antiviral therapy, wherein the compositions comprise one or more inactive ingredients and penta-O-galloyl-β-D-glucose hydrate, including any antiviral derivative, salt, hydrate, anhydrate, polymorph, or tautomer thereof. In some embodiments, the compositions comprise an amount of penta-O-galloyl-β-D-glucose hydrate, or any antiviral derivative, salt, hydrate, anhydrate, polymorph, or tautomer thereof, in an amount sufficient to inhibit a pro-inflammatory cytokine, such as IL-1α, VEGF, IL-1β, IL-2, IL-4, IL-5, IL-6, IL-8, IL-12p70, IL-13, GM-CSF, GRO, MCP-1, MIP-1α, MIP-1β, MMP-9, RANTES, and TNF-α, while inducing anti-inflammatory cytokines, such as IL-10. In some embodiments, there are provided methods of inhibiting pro-inflammatory cytokine activation and release, enhancing type 1 interferon activation, or both, and/or inducing anti-inflammatory cytokines, such as IL-10, comprising administering to a patient in need thereof an amount of penta-O-galloyl-β-D-glucose hydrate, or any antiviral derivative, salt, hydrate, anhydrate, polymorph, or tautomer thereof, sufficient to inhibit pro-inflammatory cytokine activation, release, or both. In some embodiments, there are provided antiviral and immune modulating compositions for use in methods of inhibiting pro-inflammatory cytokine activation and release, enhancing type 1 interferon activation, or both, inducing anti-inflammatory cytokines, or both, wherein the methods comprise administering to a patient in need thereof an amount of penta-O-galloyl-β-D-glucose hydrate, or any antiviral derivative, salt, hydrate, anhydrate, polymorph, or tautomer thereof, sufficient to inhibit pro-inflammatory cytokine activation, release, or both, and/or induce anti-inflammatory cytokine activation. In some embodiments, the inhibited cytokine may comprise one or more of IL-1α, VEGF, IL-1β, IL-2, IL-4, IL-5, IL-6, IL-8, IL-12p70, IL-13, GM-CSF, GRO, MCP-1, MIP-1α, MIP-1β, MMP-9, RANTES, and TNF-α, and the induced anti-inflammatory cytokine may comprise IL-10.

As described herein, helichrysetin is an antiviral compound that selectively induces apoptosis in the SARS-CoV-2 Spike (S) protein treated human macrophages, by activation of caspase 3, within 2 hours of treatment. Helichrysetin selectively inhibited pro-inflammatory cytokine, such as IL-1α, VEGF, IL-1β, IL-4, IL-5, IL-6, IL-8, IL-12p70, IL-13, MCP-1, MIP-1α, MIP-1β, MMP-9 and TNF-α, while inducing anti-inflammatory cytokines such as IL-10, in the SARS-CoV-2 Spike (S) protein treated human macrophages within 2 hours of treatment. These results indicate that the antiviral compounds enhance type 1 interferon activation, while inhibiting pro-inflammatory cytokine activation and release.

Thus, as described in more detail herein, there are provided antiviral compositions and methods of treatment, including compositions for antiviral therapy, wherein the compositions comprise one or more inactive ingredients and at least one antiviral compound, wherein the antiviral compound is helichrysetin, including any antiviral derivative, salt, hydrate, anhydrate, polymorph, or tautomer thereof. There are also provided immune modulating compositions and methods of treatment, including compositions for immune modulating and antiviral therapy, wherein the compositions comprise one or more inactive ingredients and helichrysetin, including any antiviral derivative, salt, hydrate, anhydrate, polymorph, or tautomer thereof. In some embodiments, the compositions comprise an amount of helichrysetin, or any antiviral derivative, salt, hydrate, anhydrate, polymorph, or tautomer thereof, in an amount sufficient to inhibit a pro-inflammatory cytokine, such as IL-1α, VEGF, IL-1β, IL-4, IL-5, IL-6, IL-8, IL-12p70, IL-13, MCP-1, MIP-1α, MIP-1β, MMP-9 and TNF-α, while inducing anti-inflammatory cytokines, such as IL-10. In some embodiments, there are provided methods of inhibiting pro-inflammatory cytokine activation and release, enhancing type 1 interferon activation, or both, and/or inducing anti-inflammatory cytokines, such as IL-10, comprising administering to a patient in need thereof an amount of helichrysetin, or any antiviral derivative, salt, hydrate, anhydrate, polymorph, or tautomer thereof, sufficient to inhibit pro-inflammatory cytokine activation, release, or both. In some embodiments, there are provided antiviral and immune modulating compositions for use in methods of inhibiting pro-inflammatory cytokine activation and release, enhancing type 1 interferon activation, or both, inducing anti-inflammatory cytokines, or both, wherein the methods comprise administering to a patient in need thereof an amount of helichrysetin, or any antiviral derivative, salt, hydrate, anhydrate, polymorph, or tautomer thereof, sufficient to inhibit pro-inflammatory cytokine activation, release, or both, and/or induce anti-inflammatory cytokine activation. In some embodiments, the inhibited cytokine may comprise one or more of IL-1α, VEGF, IL-1β, IL-4, IL-5, IL-6, IL-8, IL-12p70, IL-13, MCP-1, MIP-1α, MIP-1β, MMP-9 and TNF-α, and the induced anti-inflammatory cytokine may comprise IL-10.

As described herein, cinnamanilide is an antiviral compound that selectively induces apoptosis in the SARS-CoV-2 Spike (S) protein treated human macrophages, by activation of caspase 3, within 2 hours of treatment. Cinnamanilide selectively inhibited pro-inflammatory cytokine, such as IL-1α, VEGF, IL-6, IL-8, IL-13, GM-CSF, MIP-1α, MIP-1β, MMP-9 and RANTES, while inducing anti-inflammatory cytokines such as IL-10, in the SARS-CoV-2 Spike (S) protein treated human macrophages within 2 hours of treatment. These results indicate that the antiviral compounds enhance type 1 interferon activation, while inhibiting pro-inflammatory cytokine activation and release.

Thus, as described in more detail herein, there are provided antiviral compositions and methods of treatment, including compositions for antiviral therapy, wherein the compositions comprise one or more inactive ingredients and at least one antiviral compound, wherein the antiviral compound is cinnamanilide, including any antiviral derivative, salt, hydrate, anhydrate, polymorph, or tautomer thereof. There are also provided immune modulating compositions and methods of treatment, including compositions for immune modulating and antiviral therapy, wherein the compositions comprise one or more inactive ingredients and cinnamanilide, including any antiviral derivative, salt, hydrate, anhydrate, polymorph, or tautomer thereof. In some embodiments, the compositions comprise an amount of cinnamanilide, or any antiviral derivative, salt, hydrate, anhydrate, polymorph, or tautomer thereof, in an amount sufficient to inhibit a pro-inflammatory cytokine, such as IL-1α, VEGF, IL-6, IL-8, IL-13, GM-CSF, MIP-1α, MIP-1β, MMP-9 and RANTES, while inducing anti-inflammatory cytokines, such as IL-10. In some embodiments, there are provided methods of inhibiting pro-inflammatory cytokine activation and release, enhancing type 1 interferon activation, or both, and/or inducing anti-inflammatory cytokines, such as IL-10, comprising administering to a patient in need thereof an amount of cinnamanilide, or any antiviral derivative, salt, hydrate, anhydrate, polymorph, or tautomer thereof, sufficient to inhibit pro-inflammatory cytokine activation, release, or both. In some embodiments, there are provided antiviral and immune modulating compositions for use in methods of inhibiting pro-inflammatory cytokine activation and release, enhancing type 1 interferon activation, or both, inducing anti-inflammatory cytokines, or both, wherein the methods comprise administering to a patient in need thereof an amount of cinnamanilide, or any antiviral derivative, salt, hydrate, anhydrate, polymorph, or tautomer thereof, sufficient to inhibit pro-inflammatory cytokine activation, release, or both, and/or induce anti-inflammatory cytokine activation. In some embodiments, the inhibited cytokine may comprise one or more of IL-1α, VEGF, IL-6, IL-8, IL-13, GM-CSF, MIP-1α, MIP-1β, MMP-9 and RANTES, and the induced anti-inflammatory cytokine may comprise IL-10.

Helichrysetin

Helichrysetin (IUPAC name (E)-1-(2,4-dihydroxy-6-methoxyphenyl)-3-(4-hydroxyphenyl)prop-2-en-1-one) is a naturally occurring chalcone that may be extracted from the flower of helichrysum odoratissimum, having the chemical formula C₁₆H₁₄O₅, a molecular weight of 286.3 g/mol, and the following chemical structure:

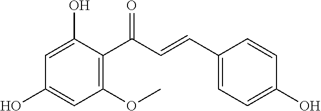 [SMILES: COc1cc(O)cc(O)c1C(=O)/C=C/c1ccc(O)cc1]

Helichrysetin

Helichrysetin inhibited expression of pro-inflammatory cytokines IL-1α, VEGF, IL-1β, IL-4, IL-5, IL-6, IL-8, IL-12p70, IL-13, MCP-1, MIP-1α, MIP-1β, MMP-9 and TNF-α in macrophages treated with Spike protein. The antiviral compound helichrysetin activated expression of cytokine modulator IL-10 in macrophages treated with Spike protein.

These results indicate that helichrysetin enhances type 1 interferon activation, while inhibiting pro-inflammatory cytokine activation and release.

As described herein helichrysetin has antiviral and immune modulating activity in vitro in the presence of SARS-CoV-2, and is expected to have antiviral and immune modulating activity in the presence of other coronaviruses, such as SARS-CoV, MERS-CoV, common cold coronavirus, as well as influenza viruses, such as influenza A and influenza B. Based on in vitro data, it is possible to enter human clinical trials, as taught in the FDA's Guidance for Industry: Antiviral Product Development—Conducting and Submitting Virology Studies to the Agency (June 2006), which is incorporated herein by reference in its entirety, especially pp. 4-5. See also McMahon et al., “New Approaches for Quantitating the Inhibition of HIV-1 Replication by Antiviral Drugs in vitro and in vivo,” Curr. Opin. Infect. Dis. 2009 December; 22(6): 574-582, which is incorporated herein in its entirety.

Where used without qualification herein, the term “helichrysetin” embraces a compound having the molecular structure above, in any form, such as solution, sol, crystal, hydrate, salt or tautomer thereof, whether crystalline or amorphous. Helichrysetin may be combined with other ingredients, such as pharmaceutically acceptable excipients. Unless otherwise specified, where a mass of helichrysetin is expressed, either alone or as part of a concentration, it is based on compound of the of the foregoing structure having a molecular weight of 286.3 g/mol.

Helichrysetin Derivatives

A helichrysetin derivative is a compound derived from helichrysetin in which one or more of the hydrogens of helichrysetin is substituted with a substituent. In some embodiments, a helichrysetin derivative is a compound of formula I in any form, such as solution, sol, crystal, hydrate, salt, or tautomer thereof, whether crystalline or amorphous.

It is expected that compounds of the following Formula I will have similar antiviral and immune modulating activity to helichrysetin, based on their structural similarity to helichrysetin, and are thus included within the antiviral and antiviral compounds as described herein.

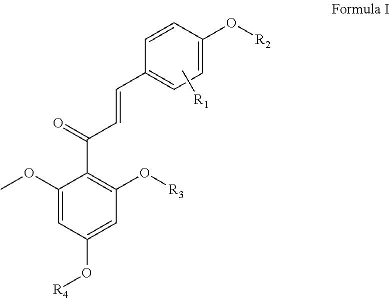

In Formula I, R₁ is H or one to three substituents, independently selected from OH, F, Cl, Br, I, CH₃, CH₂F, CHF₂, or CF₃. Each of R₂, R₃ and R₄ is H or lower alkyl, which may be otherwise unsubstituted or substituted with OH or one three substituents independently selected from F, Cl, Br, I, CH₃, CH₂F, CHF₂, or CF₃. In each case a lower alkyl group is an alkyl group of comprising one to six, preferably one to four, carbon atoms.

Compounds of Formula I in which R₁ is a substituent other than H may be prepared by electrophilic substitution on the phenyl ring by adapting art-recognized methods of electrophilic substitution. Compounds of Formula I in which R₂, R₃, and R₄ are substituents other than H may be prepared by nucleophilic substitution, by adapting art-recognized methods, wherein the oxygen of the ring hydroxy group acts as nucleophile which substitutes for a leaving group “L” in a reagent L-R₂, L-R₃, or L-R₄, respectively. Suitable leaving groups can include Cl, Br or I.

In some embodiments, the antiviral and immune modulating compound may belong to a subset of compounds of Formula I, namely compounds of Formula II:

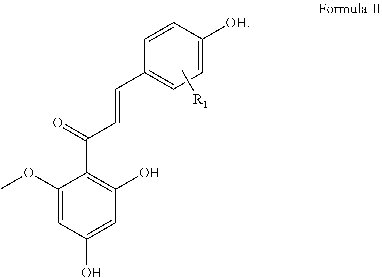

In Formula II, R₁ may be H or any of the substituents defined for R₁ in Formula I.

The effect of helichrysetin on cytokine response in human macrophages treated with CoV-2 spike protein was tested. Specifically, the effect of helichrysetin on the expression of IL-1a (interleukin 1 alpha), IL-10 (interleukin 10), IFN-γ, VEGF, IL-1β (interleukin 1 beta), IL-2 (interleukin 2), IL-4 (interleukin 4), IL-5 (interleukin 5), IL-6 (interleukin 6), IL-8 (interleukin 8), IL-12p70 (interleukin 12 p70), IL-13 (interleukin 13), GM-CSF (granulocyte-macrophage colony-stimulating factor), GRO (growth related oncogene protein), MCP-1 (monocyte chemoattractant protein-1), MIP-1α (macrophage Inflammatory Protein 1 alpha), MIP-1β (macrophage Inflammatory Protein 1 beta), MMP-9 (matrix metallopeptidase 9), RANTES (Regulated on Activation, Normal T Cell Expressed and Secreted) and TNF-α (tumor necrosis factor alpha), was detected in vitro.

Cinanserin

Cinanserin is commercially available and has the chemical formula C₂₀H₂₅ClN₂OS, a molecular weight of 376.9 g/mol, and the following chemical structure:

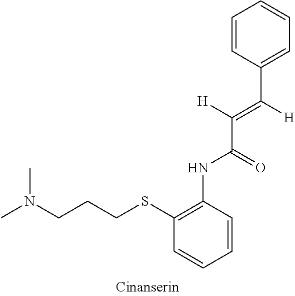 [SMILES: CN(C)CCCSc1ccccc1NC(=O)/C=C/c1ccccc1]

Where used without qualification herein, the term “cinanserin” embraces a compound having the molecular structure above, in any form, such as solution, sol, crystal, hydrate, or salt thereof, whether crystalline or amorphous. Cinnaserin is commercially available as the hydrochloride salt. Cinanserin may be combined with other ingredients, such as pharmaceutically acceptable excipients. Unless otherwise specified, where a mass of cinanserin is expressed, either alone or as part of a concentration, it is based on compound of the of the foregoing structure as the hydrochloride salt having a molecular weight of 376.9 g/mol.

Cinanserin Derivatives

A cinanserin derivative is a compound derived from cinanserin in which one or more of the hydrogens of cinanserin is substituted with a substituent. In some embodiments, a cinanserin derivative is a compound of formula III in any form, such as solution, sol, crystal, hydrate, or salt thereof, whether crystalline or amorphous.

Cinanserin Derivatives of Formula III

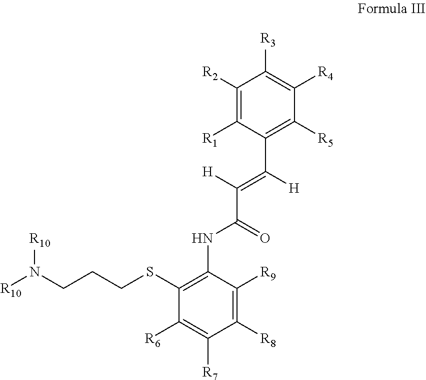

In formula III, each of R₁ to R₁₀ is H or a substituent other than H. In some embodiments, at least one of R₁ to R₁₀ is a substituent other than H. A substituent other than H may be any substituent that may be substituted for H at the indicated position of cinanserin.

In some embodiments, at least one of R₁ to R₁₀ is selected from the group consisting of halo or lower alkyl (e.g., C₁-C₆ alkyl), wherein the lower alkyl may be independently substituted by 1 to 2n+1 halo substituents, wherein n is the number of carbon atoms in the lower alkyl substituent. In some embodiments, at least one of R₁ to R₉ is F, Cl, Br, I, or a lower alkyl, e.g., methyl, ethyl, propyl, isopropyl, n-butyl, t-butyl, or s-butyl, each of which lower alkyl independently may have from 1 to 2n+1 of F, Cl, Br or In some embodiments, one of R₁ to R₉ is independently selected from methyl, ethyl, CF₃, CCl₃, CBr₃ or Cl₃ and R₁₀ is methyl or ethyl. In some embodiments, two, three, or four of R₁ to R₉ is methyl, F, Cl or CF₃. In some embodiments, at least one of R₁ to R₉ is independently selected from methyl, ethyl, CF₃, CCl₃, CBr₃ or Cl₃.

In some embodiments, the compound of formula III may be in the form of a salt. In some embodiments, the salt is a pharmaceutically acceptable salt. In some embodiments, the pharmaceutically acceptable salt is a hydrogen halide salt. In some embodiments, the hydrogen halide salt is a hydrogen chloride, hydrogen bromide or hydrogen iodide salt. In some embodiments, the pharmaceutically acceptable salt is a sulfate or tosylate salt. In some embodiments, the salt is an acetate salt.

Baicalin

Baicalin is commercially available and has chemical formula C₂₁H₁₈O₁₁, a molecular weight of 446.36 g/mol, and the following chemical structure:

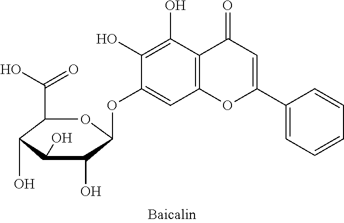

Where used without qualification herein, the term “baicalin” embraces a compound having the molecular structure above, in any form, such as solution, sol, crystal, hydrate, or salt thereof, whether crystalline or amorphous. Baicalin may be combined with other ingredients, such as pharmaceutically acceptable excipients. Unless otherwise specified, where a mass of baicalin is expressed, either alone or as part of a concentration, it is based on compound of the of the foregoing structure having a molecular weight of 446.36 g/mol.

Baicalin Derivative

A baicalin derivative is a compound derived from baicalin in which one or more of the hydrogens of baicalin is substituted with a substituent. In some embodiments, a baicalin derivative is a compound of formula IV in any form, such as solution, sol, crystal, hydrate, or salt thereof, whether crystalline or amorphous.

Baicalin Derivatives of Formula IV

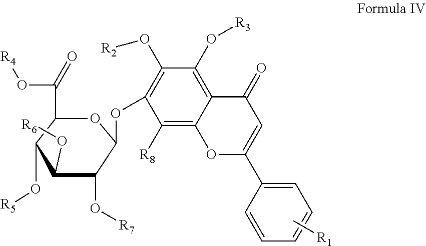

In formula IV, each of R₁ to R₈ is H or a substituent other than H. In some embodiments, at least one of R₁ to R₈ is a substituent other than H. A substituent other than H may be any substituent that may be substituted for H at the indicated position of baicalin.

In some embodiments, R₁ may be bonded at any position (o-, m-, or p-) of the phenyl ring and R₁ and R₈ independently may be H, halo (F, Cl, Br or I) or lower alkyl (e.g., C₁-C₆ alkyl), wherein in each case of R₁ or R₈, the lower alkyl may be independently substituted by hydroxy or 1 to 2n+1 halo substituents, wherein n is the number of carbon atoms in the lower alkyl substituent In some embodiments. Each of R₂ to R₇ may be H, lower alkyl (C₁-C₆) or lower acyl ((CO)C₁-C₅), wherein the lower alkyl or lower acyl may be substituted with hydroxy or from 1 to 2n+1 of F, Cl, Br. In some embodiments, each of R₂ to R₇ is independently selected from H, methyl, ethyl, CF₃, CCl₃, CBr₃, C₃, ethanoyl, COCH₂F, COCHF₂ or COCF₃, provided that at least one of R₂ to R₇ is not H. In some embodiments, each of R₂ to R₇ is H, methyl, CF₃, COCH₃ or COCF₃, or each of R₁ and R₈ is H, methyl, F, Cl, Br or CF₃, provided that at least one of R₁ to R₈ is not H.

Fangchinoline

Fangchinoline (IUPAC name (1S,14S)-9,20,25-trimethoxy-15,30-dimethyl-7,23-dioxa-15,30-diazaheptacyclo[22.6.2.2^{3,6}.1^{8,12}.1^{14,18}.0^{27,31}.0^{22,33}]hexatriaconta-3(36),4, 6(35),8,10,12(34),18,20,22(33),24,26,31-dodecaen-21-ol) is a naturally occurring alkaloid that may be extracted from Stephania tetrandra and may be obtained from various commercial sources. Fangchinoline has the chemical formula C₃₇H₄₀N₂O₆, a molecular weight of 608.72 g/mol and the following chemical structure:

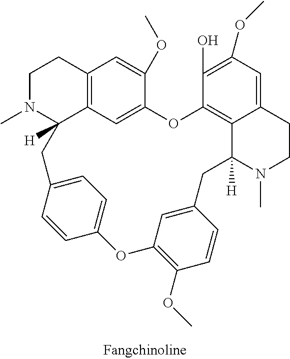 [SMILES: COc1ccc2cc1Oc1ccc(cc1)C[C@H]1c3cc(c(OC)cc3CCN1C)Oc1c(O)c(OC)cc3c1[C@H](C2)N(C)CC3]

As described herein, in addition to possessing high in vitro antiviral activity and low in vitro toxicity, fangchinoline selectively induces apoptosis in the SARS-CoV-2 Spike (S) protein treated human macrophages, by activation of caspase 3, within 2 hours of treatment.

Fangchinoline selectively inhibited pro-inflammatory cytokines, such as VEGF, IL-8, GRO, MIP-1β and MMP-9 in SARS-CoV-2 Spike (S) protein treated human macrophages within 2 hours of treatment and induced apoptosis in SARS-CoV-2 Spike (S) protein infected human macrophages through caspase 3 induction. These results indicate that fangchinoline enhances type 1 interferon activation, while inhibiting pro-inflammatory cytokine activation and release. Thus, fangchinoline has potent antiviral and immune modulating activity.

Where used without qualification herein, the term “fangchinoline” embraces a compound having the molecular structure above, in any form, such as solution, sol, crystal, hydrate, or salt thereof, whether crystalline or amorphous. Fangchinoline may be combined with other ingredients, such as pharmaceutically acceptable excipients. Unless otherwise specified, where a mass of fangchinoline is expressed, either alone or as part of a concentration, it is based on compound of the of the foregoing structure having a molecular weight of 608.72 g/mol.

Fangchinoline Derivative

A fangchinoline derivative is a compound derived from fangchinoline in which one or more of the hydrogens of fangchinoline is substituted with a substituent. In some embodiments, a fangchinoline derivative is a compound of formula V in any form, such as solution, sol, crystal, hydrate, or salt thereof, whether crystalline or amorphous.

Fangchinoline Derivatives of Formula V

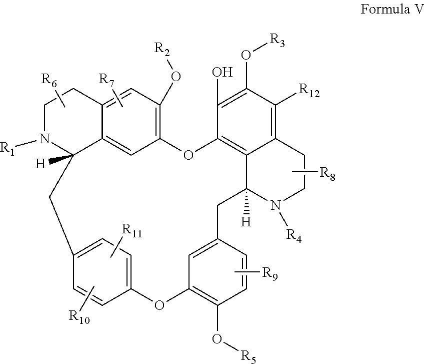

In formula V, each of R₁ to R₅ is H or lower alkyl (C₁-C₆), which may be unsubstituted or substituted hydroxy or by 1 to 2n+1 halo (F, Cl, Br, or I), wherein n is the number of carbons in the lower alkyl group. Each of R₆ to R₁₁ may be H or a substituent on any available position of the indicated ring, and may be hydroxy, lower alkyl, which may be unsubstituted or substituted with hydroxy, 1 to 2n+1 halo, or lower alkoxy, wherein the lower alkoxy may be substituted with hydroxy or 1 to 2n+1 halo, wherein n is the number of carbons in the lower alkyl or lower alkoxy. R₁₂ may be H or hydroxy, lower alkyl, which may be unsubstituted or substituted with hydroxy, 1 to 2n+1 halo, or lower alkoxy, wherein the lower alkoxy may be substituted with hydroxy or 1 to 2n+1 halo, wherein n is the number of carbons in the lower alkyl or lower alkoxy. In some embodiments, at least one of R₆ to R₁₂ is a substituent other than H. In some embodiments, at least one of R₁ to R₅ is a substituent other than methyl. In some embodiments, at least one of R₆ to R₁₂ is a substituent other than H or at least one of R₁ to R₅ is a substituent other than methyl. A substituent other than H may be any substituent that may be substituted for H at the indicated position of cepharanthine; and a substituent other than methyl may be any substituent that may be substituted for methyl at the indicated position.

As described herein fangchinoline has antiviral and immune modulating activity in vitro in the presence of SARS-CoV-2, and is expected to have antiviral and immune modulating activity in the presence of other coronaviruses, such as SARS-CoV, MERS-CoV, common cold coronavirus, as well as influenza viruses, such as influenza A and influenza B. Based on in vitro data, it is possible to enter human clinical trials, as taught in the FDA's Guidance for Industry: Antiviral Product Development—Conducting and Submitting Virology Studies to the Agency (June 2006), which is incorporated herein by reference in its entirety, especially pp. 4-5. See also McMahon et al., “New Approaches for Quantitating the Inhibition of HIV-1 Replication by Antiviral Drugs in vitro and in vivo,” Curr. Opin. Infect. Dis. 2009 December; 22(6): 574-582, which is incorporated herein in its entirety.

It is expected that compounds of the following Formula V.A will have similar antiviral and immune modulating activity to fangchinoline, based on their structural similarity to fangchinoline, and are thus included within the antiviral and antiviral compounds as described herein.

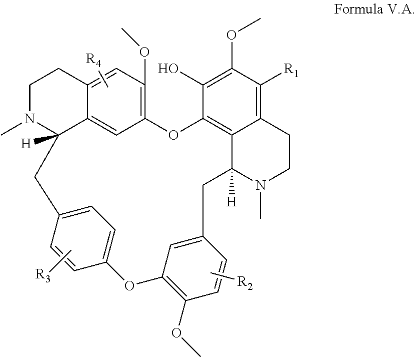

In Formula I, each of R₁ R₂, R₃ and R₄ is independently H or a substituent. R₁ is H, OH, F, Cl, Br, I, CH₃, CH₂F, CHF₂, or CF₃. R₂ and R₃ are independently H or one to three substituents, independently selected from OH, F, Cl, Br, I, CH₃, CH₂F, CHF₂, or CF₃. R₄ is H or one to two substituents, independently selected from OH, F, Cl, Br, I, CH₃, CH₂F, CHF₂, or CF₃. In some embodiments, at least one of R₁ R₂, R₃ and R₄ is a substituent.

Compounds of Formula V.A in which R₁ R₂, R₃ and R₄ is a substituent other than H may be prepared by electrophilic substitution on the aryl ring by adapting art-recognized methods of electrophilic substitution.

In some embodiments, the antiviral and immune modulating compound may belong to a subset of compounds of Formula V.A, namely compounds of Formula V.B:

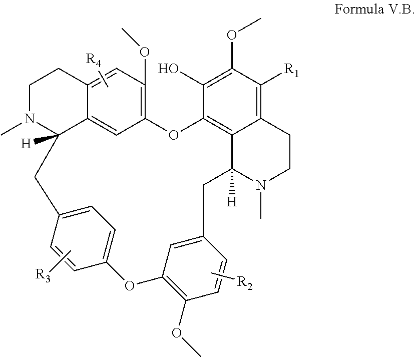

In Formula V.B, at least one of R₁ R₂, R₃ and R₄ is Cl, Br, I, CH₃, or CF₃.

The effect of fangchinoline on cytokine response in human macrophages treated with CoV-2 spike protein was tested. Specifically, the effect of fangchinoline on the expression of IL-1α (interleukin 1 alpha), IL-10 (interleukin 10), IFN-γ, VEGF, IL-1β (interleukin 1 beta), IL-2 (interleukin 2), IL-4 (interleukin 4), IL-5 (interleukin 5), IL-6 (interleukin 6), IL-8 (interleukin 8), IL-12p70 (interleukin 12 p70), IL-13 (interleukin 13), GM-CSF (granulocyte-macrophage colony-stimulating factor), GRO (growth related oncogene protein), MCP-1 (monocyte chemoattractant protein-1), MIP-1α (macrophage Inflammatory Protein 1 alpha), MIP-1β (macrophage Inflammatory Protein 1 beta), MMP-9 (matrix metallopeptidase 9), RANTES (Regulated on Activation, Normal T Cell Expressed and Secreted) and TNF-α (tumor necrosis factor alpha), was detected in vitro.

Timosaponin B is commercially available and has the chemical formula C₃₃H₅₄O₈, a molecular weight of 578.79 g/mol, and the following chemical structure:

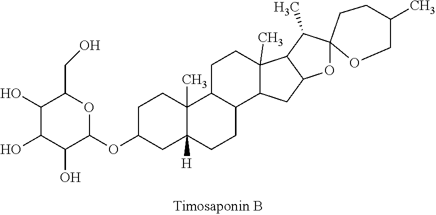 [SMILES: CC1CCC2(OC1)OC1CC3C4CC[C@@H]5CC(OC6OC(CO)C(O)C(O)C6O)CCC5(C)C4CCC3(C)C1[C@@H]2C]

Where used without qualification herein, the term “timosaponin B” embraces a compound having the molecular structure above, in any form, such as solution, sol, crystal, hydrate, or salt thereof, whether crystalline or amorphous. Timosaponin B may be combined with other ingredients, such as pharmaceutically acceptable excipients. Unless otherwise specified, where a mass of timosaponin B is expressed, either alone or as part of a concentration, it is based on compound of the of the foregoing structure having a molecular weight of 578.79 g/mol.

Timosaponin B Derivative

A timosaponin B derivative is a compound derived from timosaponin B in which one or more of the hydrogens of timosaponin B is substituted with a substituent. In some embodiments, a timosaponin B derivative is a compound of formula VI in any form, such as solution, sol, crystal, hydrate, or salt thereof, whether crystalline or amorphous.

Timosaponin B Derivatives of Formula VI

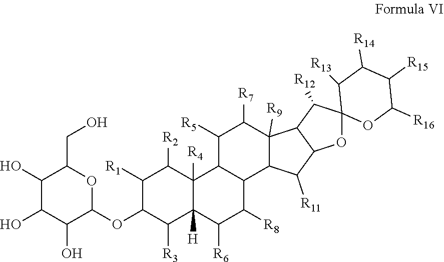

In formula VI, each of R₁ to R₁₆ is H or a substituent other than H. In some embodiments, at least one of R₁ to R₁₆ is a substituent other than H. A substituent other than H may be any substituent that may be substituted for H at the indicated position of timosaponin B.

In some embodiments, at least one of R₁ to R₁₆ is halo or lower alkyl (e.g., C₁-C₆ alkyl), wherein in each case of R₁ to R₁₆ the lower alkyl may be independently substituted by 1 to 2n+1 halo substituents, wherein n is the number of carbon atoms in the lower alkyl substituent, or a hydroxyl group. In some embodiments, at least one of R₁ to R₁₆ is OH, F, Cl, Br, I, or a lower alkyl, e.g., methyl, ethyl, propyl, isopropyl, n-butyl, t-butyl, or s-butyl, each of which lower alkyl independently may have from 1 to 2n+1 of F, Cl, Br. In some embodiments, each of R₄, R₉, R₁₂, and R₁₅ is methyl and each of the remaining substituents R₁ to R₃, R₆ to R₈, R₁₀, Ru, R₁₃, R₁₄ and R₁₆ is H, OH, F, Cl, Br, I, or a lower alkyl, e.g., methyl, ethyl, propyl, isopropyl, n-butyl, t-butyl, or s-butyl, each of which lower alkyl independently may have from 1 to 2n+1 of F, Cl, Br, provide that at least one is other than H. In some embodiments, each of R₄, R₉, R₁₂, and R₁₅ is optionally substituted lower alkyl and each of the remaining substituents R₁ to R₃, R₅ to R₈, R₁₀, Ru, R₁₃, R₁₄ and R₁₆ is H, OH, F, Cl, Br, I, or a lower alkyl, e.g., methyl, ethyl, propyl, isopropyl, n-butyl, t-butyl, or s-butyl, each of which lower alkyl independently may have from 1 to 2n+1 of F, Cl, Br, provided that at least one is other than H. In some embodiments, each of R₄, R₉, R₁₂, and R₁₅ is methyl, CH₂F, CHF₂ or CF₃, and each of the remaining substituents R₁ to R₃, R₅ to R₈, R₁₀, R₁₁, R₁₃, R₁₄ and R₁₆ is H, OH, F, Cl, Br, I, or a lower alkyl, e.g., methyl, ethyl, propyl, isopropyl, n-butyl, t-butyl, or s-butyl, each of which lower alkyl independently may have from 1 to 2n+1 of F, Cl, Br, provided that at least one is other than H.

Cepharanthine

Cepharanthine (IUPAC name (14S,27S)-22,33-dimethoxy-13,28-dimethyl-2,5,7,20-tetraoxa-13,28-diazaoctacyclo[25.6.2.2^{16,19}.1^{3,10}.1^{21,25}.0^{4,8}.0^{31,35}.0^{14,39}]nonatri-aconta-1(33),3(39),4(8),9,16(38),17,19(37),21,23,25(36),31,34-dodecaene) is a commercially available alkaloid compound, which may be isolated from tubers of Stephania and has the chemical formula C₃₇H₃₈N₂O₆, a molecular weight of 606.71 g/mol and the following chemical structure:

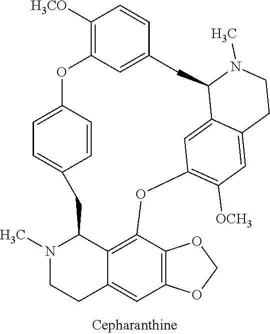 [SMILES: COc1ccc2cc1Oc1ccc(cc1)C[C@H]1c3c(cc4c(c3Oc3cc5c(cc3OC)CCN(C)[C@@H]5C2)OCO4)CCN1C]

Where used without qualification herein, the term “cepharanthine” embraces a compound having the molecular structure above, in any form, such as solution, sol, crystal, hydrate, or salt thereof, whether crystalline or amorphous. Cepharanthine may be combined with other ingredients, such as pharmaceutically acceptable excipients. Unless otherwise specified, where a mass of cepharanthine is expressed, either alone or as part of a concentration, it is based on compound of the of the foregoing structure having a molecular weight of 606.71 g/mol.

Cepharanthine selectively inhibited pro-inflammatory cytokines, such as VEGF, IL-6, IL-8, GRO, MIP-1α, MIP-1β and MMP-9 while inducing anti-inflammatory cytokines such as IL-10 in SARS-CoV-2 Spike (S) protein treated human macrophages within 2 hours of treatment. Cepharanthine also suppressed multiple pro-inflammatory cytokines such as IL-1β, TNF-α, IL-6 IL-8, IL-12, MCP-1 and MIP-1α in SARS-CoV-2 Spike (S) protein treated human macrophages, while increasing expression of the anti-inflammatory cytokine IL-10 in nanomolar concentrations, within 2 hours of treatment, without compromising cell viability. Cepharanthine is an entry inhibitor, reducing plasma membrane fluidity interfering with the virus' ability to attach to ACE2 receptor and RNA synthesis inhibitor, inhibiting viral replication.

These results indicate that cepharanthine inhibits SARS-CoV-2 viral entry into human cells and inhibits viral replication, thereby neutralizing SARS-CoV-2 infection and enhancing type 1 interferon activation, while inhibiting pro-inflammatory cytokine activation and release

As described herein cepharanthine has antiviral and immune modulating activity in vitro in the presence of SARS-CoV-2, and is expected to have antiviral and immune modulating activity in the presence of other coronaviruses, such as SARS-CoV, MERS-CoV, common cold coronavirus, as well as influenza viruses, such as influenza A and influenza B. Based on in vitro data, it is possible to enter human clinical trials, as taught in the FDA's Guidance for Industry: Antiviral Product Development—Conducting and Submitting Virology Studies to the Agency (June 2006), which is incorporated herein by reference in its entirety, especially pp. 4-5. See also McMahon et al., “New Approaches for Quantitating the Inhibition of HIV-1 Replication by Antiviral Drugs in vitro and in vivo,” Curr. Opin. Infect. Dis. 2009 December; 22(6): 574-582, which is incorporated herein in its entirety.

Cepharanthine Derivatives

A cepharanthine derivative is a compound derived from cepharanthine in which one or more of the hydrogens of cepharanthine is substituted with a substituent. In some embodiments, a cepharanthine derivative is a compound of formula VII in any form, such as solution, sol, crystal, hydrate, or salt thereof, whether crystalline or amorphous.

Cepharanthine Derivatives of Formula VII

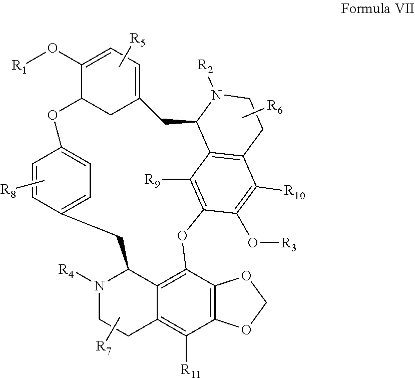

In formula VII, each of R₁ to R₄ is H, lower alkyl (C₁-C₆), which may be unsubstituted or substituted by 1 to 2n+1 halo (F, Cl, Br, or I), wherein n is the number of carbons in the lower alkyl group, or hydroxy. Each of R₅ to R₈ may be H or a substituent on any available position of the indicated ring, and may be hydroxy, lower alkyl, which may be unsubstituted or substituted with hydroxy, 1 to 2n+1 halo, or lower alkoxy, wherein the lower alkoxy may be substituted with hydroxy or 1 to 2n+1 halo, wherein n is the number of carbons in the lower alkyl or lower alkoxy. Each of R₉ to R₁₁ may be H, hydroxy, lower alkyl, which may be unsubstituted or substituted with hydroxy, 1 to 2n+1 halo, or lower alkoxy, wherein the lower alkoxy may be substituted with hydroxy or 1 to 2n+1 halo, wherein n is the number of carbons in the lower alkyl or lower alkoxy. In some embodiments, at least one of R₅ to R₁₁ is a substituent other than H. In some embodiments, at least one of R₁ to R₄ is a substituent other than methyl. In some embodiments, at least one of R₅ to R₁₁ is a substituent other than H or at least one of R₁ to R₄ is a substituent other than methyl. A substituent other than H may be any substituent that may be substituted for H at the indicated position of cepharanthine; and a substituent other than methyl may be any substituent that may be substituted for methyl at the indicated position.

It is expected that compounds of the following Formula VII.A will have similar immune modulating activity to cepharanthine, based on their structural similarity to cepharanthine, and are thus included within the antiviral compounds as described herein.

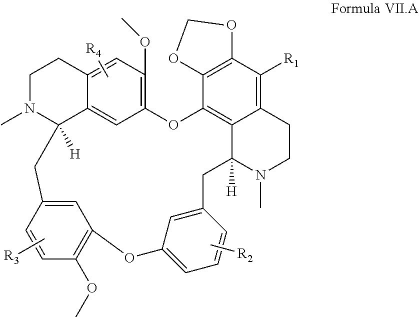

In Formula VILA, each of R₁ R₂, R₃ and R₄ is independently H or a substituent. R₁ is H, OH, F, Cl, Br, I, CH₃, CH₂F, CHF₂, or CF₃. R₂ and R₃ are independently H or one to three substituents, independently selected from OH, F, Cl, Br, I, CH₃, CH₂F, CHF₂, or CF₃. R₄ is H or one to two substituents, independently selected from OH, F, Cl, Br, I, CH₃, CH₂F, CHF₂, or CF₃. In some embodiments, at least one of R₁ R₂, R₃ and R₄ is a substituent.

Compounds of Formula VII.A in which R₁ R₂, R₃ and R₄ is a substituent other than H may be prepared by electrophilic substitution on the aryl ring by adapting art-recognized methods of electrophilic substitution.

In some embodiments, the immune modulating compound may belong to a subset of compounds of Formula VILA, namely compounds of Formula VII.B:

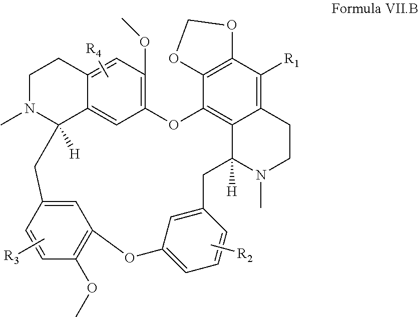

In Formula VII.B, at least one of R₁ R₂, R₃ and R₄ is Cl, Br, I, CH₃, or CF₃.

The effect of cepharanthine on cytokine response in human macrophages treated with CoV-2 spike protein was tested. Specifically, the effect of cepharanthine on the expression of IL-1α (interleukin 1 alpha), IL-10 (interleukin 10), IFN-γ, VEGF, IL-1β (interleukin 1 beta), IL-2 (interleukin 2), IL-4 (interleukin 4), IL-5 (interleukin 5), IL-6 (interleukin 6), IL-8 (interleukin 8), IL-12p70 (interleukin 12 p70), IL-13 (interleukin 13), GM-CSF (granulocyte-macrophage colony-stimulating factor), GRO (growth related oncogene protein), MCP-1 (monocyte chemoattractant protein-1), MIP-1α (macrophage Inflammatory Protein 1 alpha), MIP-1β (macrophage Inflammatory Protein 1 beta), MMP-9 (matrix metallopeptidase 9), RANTES (Regulated on Activation, Normal T Cell Expressed and Secreted) and TNF-α (tumor necrosis factor alpha), was detected in vitro.

Tetrandrine

Tetrandrine (IUPAC name (1S,14S)-9,20,21,25-tetramethoxy-15,30-dimethyl-7,23-dioxa-15,30-diazaheptacyclo[22.6.2.2^{3,6}.1^{8,12}.1^{14,18}.0^{27,31}.0^{22,33}]hexatriaconta-3(36),4,6(35),8,10,12(34),18,20,22(33),24,26,31-dodecaene) is a commercially available alkaloid compound, which may be isolated from t Stephania tetrandra and has the chemical formula C₃₈H₄₂N₂O₆, a molecular weight of 622.7 g/mol and the following chemical structure:

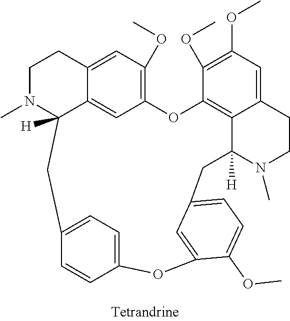 [SMILES: COc1ccc2cc1Oc1ccc(cc1)C[C@H]1c3cc(c(OC)cc3CCN1C)Oc1c(OC)c(OC)cc3c1[C@H](C2)N(C)CC3]

Where used without qualification herein, the term “tetrandrine” embraces a compound having the molecular structure above, in any form, such as solution, sol, crystal, hydrate, or salt thereof, whether crystalline or amorphous. Tetrandrine may be combined with other ingredients, such as pharmaceutically acceptable excipients. Unless otherwise specified, where a mass of tetrandrine is expressed, either alone or as part of a concentration, it is based on compound of the of the foregoing structure having a molecular weight of 622.7 g/mol.

Tetrandrine is a proteolytic processing inhibitor of S1/S2 site of the coronavirus spike protein, attaching to the human angiotensin converting enzyme 2 receptor (ACE2).

These results indicate that cepharanthine inhibits SARS-CoV-2 viral entry into human cells and inhibits viral replication, thereby neutralizing SARS-CoV-2 infection while also enhance type 1 interferon activation and inhibiting pro-inflammatory cytokine activation and release.

As described herein tetrandrine has antiviral and immune modulating activity in vitro in the presence of SARS-CoV-2, and is expected to have antiviral and immune modulating activity in the presence of other coronaviruses, such as SARS-CoV, MERS-CoV, common cold coronavirus, as well as influenza viruses, such as influenza A and influenza B. Based on in vitro data, it is possible to enter human clinical trials, as taught in the FDA's Guidance for Industry: Antiviral Product Development—Conducting and Submitting Virology Studies to the Agency (June 2006), which is incorporated herein by reference in its entirety, especially pp. 4-5. See also McMahon et al., “New Approaches for Quantitating the Inhibition of HIV-1 Replication by Antiviral Drugs in vitro and in vivo,” Curr. Opin. Infect. Dis. 2009 December; 22(6): 574-582, which is incorporated herein in its entirety.

The effect of tetrandrine on cytokine response in human macrophages treated with CoV-2 spike protein was tested. Specifically, the effect of tetrandrine on the expression of IL-1α (interleukin 1 alpha), IL-10 (interleukin 10), IFN-γ, VEGF, IL-1β (interleukin 1 beta), IL-2 (interleukin 2), IL-4 (interleukin 4), IL-5 (interleukin 5), IL-6 (interleukin 6), IL-8 (interleukin 8), IL-12p70 (interleukin 12 p70), IL-13 (interleukin 13), GM-CSF (granulocyte-macrophage colony-stimulating factor), GRO (growth related oncogene protein), MCP-1 (monocyte chemoattractant protein-1), MIP-1α (macrophage Inflammatory Protein 1 alpha), MIP-1β (macrophage Inflammatory Protein 1 beta), MMP-9 (matrix metallopeptidase 9), RANTES (Regulated on Activation, Normal T Cell Expressed and Secreted) and TNF-α (tumor necrosis factor alpha), was detected in vitro.

Tetrandrine Derivatives

A tetrandrine derivative is a compound derived from tetrandrine in which one or more of the hydrogens of tetrandrine is substituted with a substituent. In some embodiments, a tetrandrine derivative is a compound of formula VIII in any form, such as solution, sol, crystal, hydrate, or salt thereof, whether crystalline or amorphous.

Tetrandrine Derivatives of Formula VIII

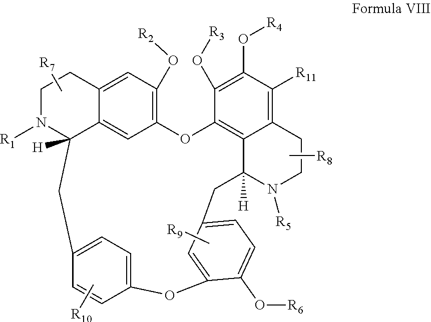

In formula VIII, each of R₁ to R₆ is lower alkyl (C₁-C₆), which may be unsubstituted or substituted by 1 to 2n+1 halo (F, Cl, Br, or I), wherein n is the number of carbons in the lower alkyl group, or hydroxy. Each of R₇ to R₁₀ may be H a substituent on any available position of the indicated ring, and may be hydroxy, lower alkyl, which may be unsubstituted or substituted with hydroxy, 1 to 2n+1 halo, or lower alkoxy, wherein the lower alkoxy may be substituted with hydroxy or 1 to 2n+1 halo, wherein n is the number of carbons in the lower alkyl or lower alkoxy. R₁₁ may be H, hydroxy, lower alkyl, which may be unsubstituted or substituted with hydroxy, 1 to 2n+1 halo, or lower alkoxy, wherein the lower alkoxy may be substituted with hydroxy or 1 to 2n+1 halo, wherein n is the number of carbons in the lower alkyl or lower alkoxy. In some embodiments, at least one of R₇ to R₁₁ is a substituent other than H. In some embodiments, at least one of R₁ to R₆ is a substituent other than methyl. In some embodiments, at least one of R₇ to R₁₁ is a substituent other than H or at least one of R₁ to R₆ is a substituent other than methyl. A substituent other than H may be any substituent that may be substituted for H at the indicated position of tetrandrine; and a substituent other than methyl may be any substituent that may be substituted for methyl at the indicated position.

It is expected that compounds of the following Formula VIII.A will have similar antiviral and immune modulating activity to tetrandrine, based on their structural similarity to tetrandrine, and are thus included within the antiviral compound as described herein.

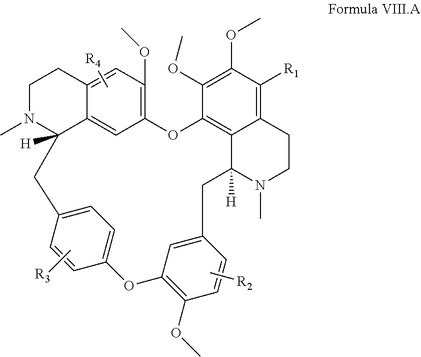

In Formula VIII.A, each of R₁ R₂, R₃ and R₄ is independently H or a substituent. R₁ is H, OH, F, Cl, Br, I, CH₃, CH₂F, CHF₂, or CF₃. R₂ and R₃ are independently H or one to three substituents, independently selected from OH, F, Cl, Br, I, CH₃, CH₂F, CHF₂, or CF₃. R₄ is H or one to two substituents, independently selected from OH, F, Cl, Br, I, CH₃, CH₂F, CHF₂, or CF₃. In some embodiments, at least one of R₁ R₂, R₃ and R₄ is a substituent.

Compounds of Formula I in which R₁ R₂, R₃ and R₄ is a substituent other than H may be prepared by electrophilic substitution on the aryl ring by adapting art-recognized methods of electrophilic substitution.

In some embodiments, the antiviral compound may belong to a subset of compounds of Formula VIII.A, namely compounds of Formula VIII.B:

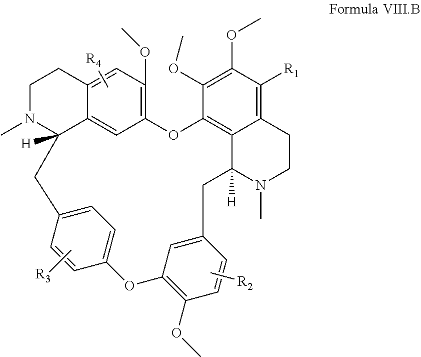

In Formula VIII.B, at least one of R₁ R₂, R₃ and R₄ is Cl, Br, I, CH₃, or CF₃.

Bavachalcone B

Bavachalcone B is a commercially available, naturally occurring chalcone that may be extracted from Psoralea corylifolia L and has the chemical formula C₂₀H₂₀O₄, a molecular weight of 324.37 g/mol and the following chemical structure:

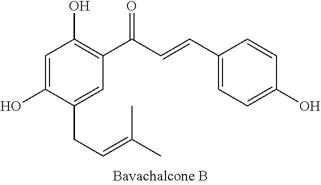 [SMILES: CC(C)=CCc1cc(C(=O)/C=C/c2ccc(O)cc2)c(O)cc1O]

Where used without qualification herein, the term “bavachalcone B” embraces a compound having the molecular structure above, in any form, such as solution, sol, crystal, hydrate, or salt thereof, whether crystalline or amorphous. Bavachalcone B may be combined with other ingredients, such as pharmaceutically acceptable excipients. Unless otherwise specified, where a mass of bavachalcone B is expressed, either alone or as part of a concentration, it is based on compound of the of the foregoing structure having a molecular weight of 324.37 g/mol.

Bavachalcone B Derivatives

A bavachalcone B derivative is a compound derived from bavachalcone B in which one or more of the hydrogens of bavachalcone B is substituted with a substituent. In some embodiments, a bavachalcone B derivative is a compound of formula IX in any form, such as solution, sol, crystal, hydrate, or salt thereof, whether crystalline or amorphous.

Bavachalcone B Derivatives of Formula IX

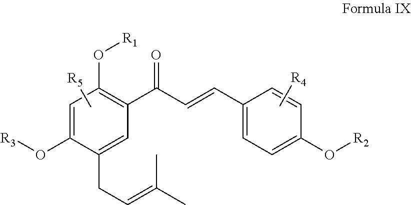

In formula IX, each of R₁ to R₃ is H or lower alkyl (C₁-C₆), which may be unsubstituted or substituted hydroxy or by 1 to 2n+1 halo (F, Cl, Br, or I), wherein n is the number of carbons in the lower alkyl group. R₄ and R₅ independently may be H or a substituent on any available position of the indicated ring, and may be hydroxy, lower alkyl, which may be unsubstituted or substituted with hydroxy, 1 to 2n+1 halo, or lower alkoxy, wherein the lower alkoxy may be substituted with hydroxy or 1 to 2n+1 halo, wherein n is the number of carbons in the lower alkyl or lower alkoxy. In some embodiments, at least one of R₄ or R₅ is a substituent other than H. In some embodiments, at least one of R₁ to R₃ is a substituent other than methyl. In some embodiments, at least one of R₄ or R₅ is a substituent other than H or at least one of R₁ to R₃ is a substituent other than methyl. A substituent other than H may be any substituent that may be substituted for H at the indicated position of bavachalcone B; and a substituent other than methyl may be any substituent that may be substituted for methyl at the indicated position.

Rosmarinic Acid

Rosmarinic acid is a commercially available, naturally occurring chalcone that may be extracted from Rosemarinus officinalis L and has the chemical formula C₁₈H₁₁O₈, a molecular weight of 360.3 g/mol and the following chemical structure:

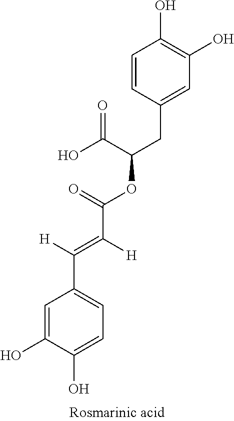 [SMILES: O=C(/C=C/c1ccc(O)c(O)c1)O[C@H](Cc1ccc(O)c(O)c1)C(=O)O]

Where used without qualification herein, the term “rosmarinic acid” embraces a compound having the molecular structure above, in any form, such as solution, sol, crystal, hydrate, or salt thereof, whether crystalline or amorphous. rosmarinic acid may be combined with other ingredients, such as pharmaceutically acceptable excipients. Unless otherwise specified, where a mass of rosmarinic acid is expressed, either alone or as part of a concentration, it is based on compound of the of the foregoing structure having a molecular weight of 360.3 g/mol.

Rosmarinic Acid Derivative

A rosmarinic acid derivative is a compound derived from rosmarinic acid in which one or more of the hydrogens of rosmarinic acid is substituted with a substituent. In some embodiments, a rosmarinic acid derivative is a compound of formula X in any form, such as solution, sol, crystal, hydrate, or salt thereof, whether crystalline or amorphous.

Rosmarinic Acid Derivatives of Formula X

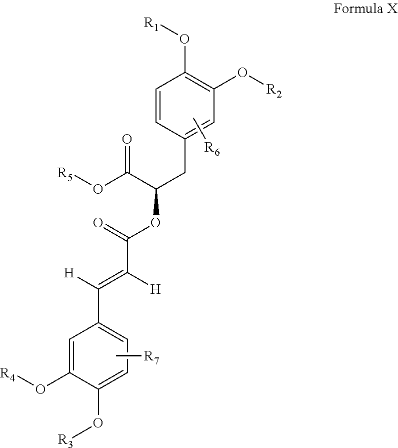

In Formula X, each of R₁ to R₅ is H, lower acyl (—CO(C₂-C₅)), which may be unsubstituted or substituted by hydroxy, lower alkoxy (C₁-C₆), optionally having a hydroxy or 1 to 2n+1 halo (F, Cl, Br, or I) substituents, or lower alkyl (C₁-C₆), which may be unsubstituted or substituted by hydroxy or by 1 to 2n+1 halo (F, Cl, Br, or I), wherein n is the number of carbons in the lower alkyl group. R₆ and R₇ independently may be H or a substituent on any available position of the indicated ring, and may be hydroxy, lower alkyl, which may be unsubstituted or substituted with hydroxy, 1 to 2n+1 halo, or lower alkoxy, wherein the lower alkoxy may be substituted with hydroxy or 1 to 2n+1 halo, wherein n is the number of carbons in the lower alkyl or lower alkoxy. In some embodiments, at least one of R₁ or R₇ is a substituent other than H. A substituent other than H may be any substituent that may be substituted for H at the indicated position of rosmarinic acid.

Formononetin

As described herein, formononetin is an antiviral compound that selectively induces apoptosis in the SARS-CoV-2 Spike (S) protein treated human macrophages, by activation of caspase 3, within 2 hours of treatment.

Formononetin selectively inhibited pro-inflammatory cytokine, such as VEGF, IL-6, IL-8, GRO, MCP-1, MIP-1α, MIP-1β, MMP-9 and RANTES, while inducing anti-inflammatory cytokines such as IL-10 and IFN-γ, in the SARS-CoV-2 Spike (S) protein treated human macrophages within 2 hours of treatment.

These results indicate that the antiviral compounds enhance type 1 interferon activation, while inhibiting pro-inflammatory cytokine activation and release.

Formononetin (IUPAC name 7-hydroxy-3-(4-methoxyphenyl)chromen-4-one) has the chemical structure:

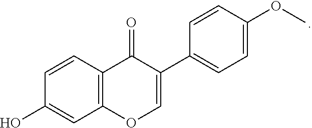 [SMILES: COc1ccc(-c2coc3cc(O)ccc3c2=O)cc1]

Formononetin may be isolated from various herbs and plants, such as red clover and legumes and is commercially available from various sources.

Formononetin inhibited expression of pro-inflammatory cytokines VEGF, IL-6, IL-8, GRO, MCP-1, MIP-1α, MIP-1β, MMP-9 and RANTES in macrophages treated with Spike protein. The antiviral compound formononetin also activated expression of cytokine modulators IL-10 and IFN-γ in macrophages treated with Spike protein. Additionally, formononetin induced apoptosis in macrophages treated with SARS-CoV-2 spike protein by caspase 3 activation.

These results indicate that formononetin enhances type 1 interferon activation, while inhibiting pro-inflammatory cytokine activation and release.

As described herein formononetin has immune modulating activity in vitro in the presence of SARS-CoV-2, and is expected to have immune modulating activity in the presence of other coronaviruses, such as SARS-CoV, MERS-CoV, common cold coronavirus, as well as influenza viruses, such as influenza A and influenza B. Based on in vitro data, it is possible to enter human clinical trials, as taught in the FDA's Guidance for Industry: Antiviral Product Development—Conducting and Submitting Virology Studies to the Agency (June 2006), which is incorporated herein by reference in its entirety, especially pp. 4-5. See also McMahon et al., “New Approaches for Quantitating the Inhibition of HIV-1 Replication by Antiviral Drugs in vitro and in vivo,” Curr. Opin. Infect. Dis. 2009 December; 22(6): 574-582, which is incorporated herein in its entirety.

It is expected that compounds of the following Formula XI, below, will have similar immune modulating activity to formononetin, based on their structural similarity to formononetin, and are thus included within the antiviral compounds as described herein.

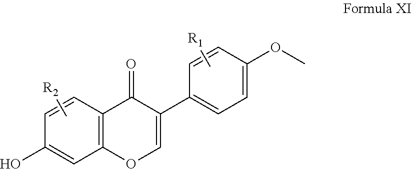

In Formula XI, each of R₁ and R₂ is H or one to three substituents, independently selected from OH, F, Cl, Br, I, CH₃, CH₂F, CHF₂, or CF₃.

Compounds of Formula XI in which R₁ or R₂ is a substituent other than H may be prepared by electrophilic substitution on the aryl ring by adapting art-recognized methods of electrophilic substitution.

In some embodiments, the immune modulating compound may belong to a subset of compounds of Formula I, namely compounds of Formula XI.A:

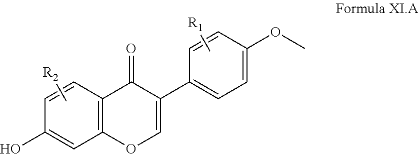

In Formula XI.A, R₁ may be H, Cl, Br, I, CH₃, or CF₃.

The effect of formononetin on cytokine response in human macrophages treated with CoV-2 spike protein was tested. Specifically, the effect of formononetin on the expression of IL-1α (interleukin 1 alpha), IL-10 (interleukin 10), IFN-γ, VEGF, IL-1β (interleukin 1 beta), IL-2 (interleukin 2), IL-4 (interleukin 4), IL-5 (interleukin 5), IL-6 (interleukin 6), IL-8 (interleukin 8), IL-12p70 (interleukin 12 p70), IL-13 (interleukin 13), GM-CSF (granulocyte-macrophage colony-stimulating factor), GRO (growth related oncogene protein), MCP-1 (monocyte chemoattractant protein-1), MIP-1α (macrophage Inflammatory Protein 1 alpha), MIP-1β (macrophage Inflammatory Protein 1 beta), MMP-9 (matrix metallopeptidase 9), RANTES (Regulated on Activation, Normal T Cell Expressed and Secreted) and TNF-α (tumor necrosis factor alpha), was detected in vitro.

Baicalein

Baicalein (IUPAC name 5,6,7-trihydroxy-2-phenylchromen-4-one) has the chemical structure:

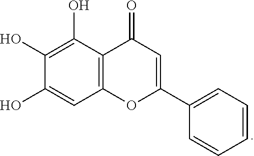 [SMILES: O=c1cc(-c2ccccc2)oc2cc(O)c(O)c(O)c12]

Baicalein was originally isolated from the roots of Scutellaria baicalensis and Scutellaria lateriflora and is commercially available from various sources.

Baicalein potently inhibited expression of pro-inflammatory cytokines IL-1α, IL-8, GRO, MCP-1, MIP-1α, MIP-1β, MMP-9 and TNF-α in macrophages treated with Spike protein. The antiviral compound baicalein activated expression of cytokine modulators IL-10 and IFN-γ in macrophages treated with Spike protein. Additionally, baicalein induced apoptosis in macrophages treated with SARS-CoV-2 spike protein by caspase 3 activation.

These results indicate that baicalein enhances type 1 interferon activation, while inhibiting pro-inflammatory cytokine activation and release.

As described herein baicalein has antiviral and immune modulating activity in vitro in the presence of SARS-CoV-2, and is expected to have antiviral and immune modulating activity in the presence of other coronaviruses, such as SARS-CoV, MERS-CoV, common cold coronavirus, as well as influenza viruses, such as influenza A and influenza B. Based on in vitro data, it is possible to enter human clinical trials, as taught in the FDA's Guidance for Industry: Antiviral Product Development—Conducting and Submitting Virology Studies to the Agency (June 2006), which is incorporated herein by reference in its entirety, especially pp. 4-5. See also McMahon et al., “New Approaches for Quantitating the Inhibition of HIV-1 Replication by Antiviral Drugs in vitro and in vivo,” Curr. Opin. Infect. Dis. 2009 December; 22(6): 574-582, which is incorporated herein in its entirety.

It is expected that compounds of the following Formula XII will have similar antiviral and immune modulating activity to baicalein, based on their structural similarity to baicalein, and are thus included within the antiviral compounds as described herein.

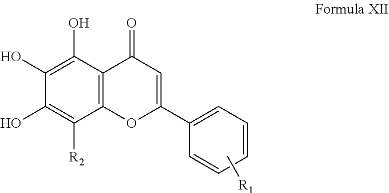

In Formula XII, R₁ is H or one to three substituents, independently selected from OH, F, Cl, Br, I, CH₃, CH₂F, CHF₂, or CF₃.

Compounds of Formula XII in which R₁ is a substituent other than H may be prepared by electrophilic substitution on the aryl ring by adapting art-recognized methods of electrophilic substitution.

In some embodiments, the antiviral compound may belong to a subset of compounds of Formula I, namely compounds of Formula XII.A:

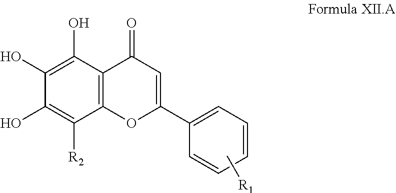

In Formula XII.A, R₁ is Cl, Br, I, CH₃, or CF₃.

The effect of baicalein on cytokine response in human macrophages treated with CoV-2 spike protein was tested. Specifically, the effect of baicalein on the expression of IL-1α (interleukin 1 alpha), IL-10 (interleukin 10), IFN-γ, VEGF, IL-1B (interleukin 1 beta), IL-2 (interleukin 2), IL-4 (interleukin 4), IL-5 (interleukin 5), IL-6 (interleukin 6), IL-8 (interleukin 8), IL-12p70 (interleukin 12 p70), IL-13 (interleukin 13), GM-CSF (granulocyte-macrophage colony-stimulating factor), GRO (growth related oncogene protein), MCP-1 (monocyte chemoattractant protein-1), MIP-1α (macrophage Inflammatory Protein 1 alpha), MIP-1β (macrophage Inflammatory Protein 1 beta), MMP-9 (matrix metallopeptidase 9), RANTES (Regulated on Activation, Normal T Cell Expressed and Secreted) and TNF-α (tumor necrosis factor alpha), was detected in vitro.

### Example 6D: Kazinol A

Kazinol A (IUPAC name 4-[(2S)-7-hydroxy-3,4-dihydro-2H-chromen-2-yl]-3,6-bis(3-methylbut-2-enyl)benzene-1,2-diol) has the chemical structure:

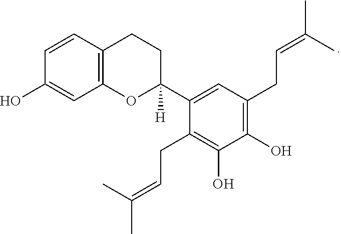 [SMILES: CC(C)=CCc1cc([C@@H]2CCc3ccc(O)cc3O2)c(CC=C(C)C)c(O)c1O]

Kazinol A is commercially available from various sources.

Kazinol A potently inhibited expression of pro-inflammatory cytokines IL-1α, IL-4, IL-8, MIP-1β and MMP-9 in macrophages treated with Spike protein. The antiviral compound kazinol A activated expression of cytokine modulator IL-10 in macrophages treated with Spike protein. Additionally, kazinol A induced apoptosis in macrophages treated with SARS-CoV-2 spike protein by caspase 3 activation.

These results indicate that kazinol A is a potent antiviral compound and immune modulator.

As described herein kazinol A has antiviral and immune modulating activity in vitro in the presence of SARS-CoV-2, and is expected to have antiviral and immune modulating activity in the presence of other coronaviruses, such as SARS-CoV, MERS-CoV, common cold coronavirus, as well as influenza viruses, such as influenza A and influenza B. Based on in vitro data, it is possible to enter human clinical trials, as taught in the FDA's Guidance for Industry: Antiviral Product Development—Conducting and Submitting Virology Studies to the Agency (June 2006), which is incorporated herein by reference in its entirety, especially pp. 4-5. See also McMahon et al., “New Approaches for Quantitating the Inhibition of HIV-1 Replication by Antiviral Drugs in vitro and in vivo,” Curr. Opin. Infect. Dis. 2009 December; 22(6): 574-582, which is incorporated herein in its entirety.

It is expected that compounds of the following Formula XIII will have similar antiviral and immune modulating activity to kazinol A, based on their structural similarity to kazinol A, and are thus included within the antiviral compounds as described herein.

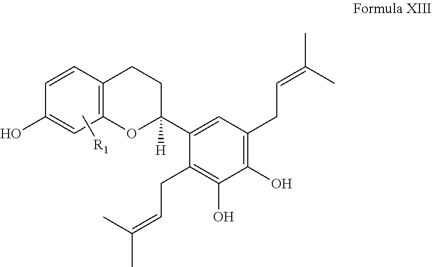

In Formula XIII, R₁ is H or one to three substituents, independently selected from OH, F, Cl, Br, I, CH₃, CH₂F, CHF₂, or CF₃.

Compounds of Formula XIII in which R₁ is a substituent other than H may be prepared by electrophilic substitution on the aryl ring by adapting art-recognized methods of electrophilic substitution.

In some embodiments, the antiviral and immune modulating compound may belong to a subset of compounds of Formula XIII namely compounds of Formula XIII.A:

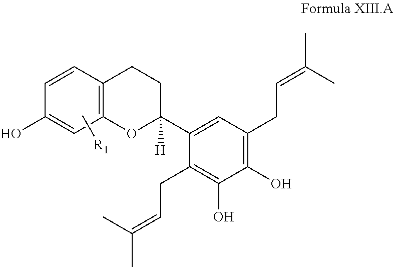

In Formula XIII.A, R₁ is Cl, Br, I, CH₃, or CF₃.

The effect of kazinol A on cytokine response in human macrophages treated with CoV-2 spike protein was tested. Specifically, the effect of kazinol A on the expression of IL-1α (interleukin 1 alpha), IL-10 (interleukin 10), IFN-γ, VEGF, IL-1β (interleukin 1 beta), IL-2 (interleukin 2), IL-4 (interleukin 4), IL-5 (interleukin 5), IL-6 (interleukin 6), IL-8 (interleukin 8), IL-12p70 (interleukin 12 p70), IL-13 (interleukin 13), GM-CSF (granulocyte-macrophage colony-stimulating factor), GRO (growth related oncogene protein), MCP-1 (monocyte chemoattractant protein-1), MIP-1α (macrophage Inflammatory Protein 1 alpha), MIP-1β (macrophage Inflammatory Protein 1 beta), MMP-9 (matrix metallopeptidase 9), RANTES (Regulated on Activation, Normal T Cell Expressed and Secreted) and TNF-α (tumor necrosis factor alpha), was detected in vitro.

Penta-O-Galloyl-β-D-Glucose Hydrate

The adapter protein myeloid differentiation primary response 88 (MyD88), responds to PAMPs signaling on PRRs. MyD88 signaling can lead to the production of pro- or anti-inflammatory cytokines as well as type I interferons. Type 1 interferons are desired in the control of viral infections. Distinct pathways downstream of interleukin 1 receptor (IL-1R) associated kinase (IRAK) family members in association with MyD88 regulate these outputs, and the outcome of signaling can be influenced by the cell type and location of signal initiation.

One skilled in the art will readily recognize that penta-O-galloyl-β-D-glucose hydrate is a solid form of penta-O-galloyl-β-D-glucose and that when penta-O-galloyl-β-D-glucose is dissolved in an aqueous solvent, the solution contains penta-O-galloyl-β-D-glucose as a solute. In aqueous solution, as well as in vivo, penta-O-galloyl-β-D-glucose is the physical form of the solute and is the biologically active form. Additionally, it will be recognized that in solid form penta-O-galloyl-β-D-glucose hydrate may exist with a variable number of waters of hydration. Thus, in the formula penta-O-galloyl-β-D-glucose xH₂O, x may be 0 (i.e., penta-O-galloyl-β-D-glucose is anhydrous) or a real number greater than 0. As penta-O-galloyl-β-D-glucose is hygroscopic, the value of x may vary over time for a sample of penta-O-galloyl-β-D-glucose exposed to an ambient atmosphere, especially for a sample of pure penta-O-galloyl-β-D-glucose hydrate (i.e., not combined with one or more stabilizing excipients). One skilled in the art will recognize, however, that the immune modulating moiety in penta-O-galloyl-β-D-glucose hydrate is the penta-O-galloyl-β-D-glucose portion itself—that is, the degree of hydration is not expected to affect the immune modulating nature of penta-O-galloyl-β-D-glucose, though the degree of hydration should be taken into account when preparing formulations containing penta-O-galloyl-β-D-glucose. Thus, wherever penta-O-galloyl-β-D-glucose hydrate is in an aqueous solution (e.g., an in vitro macrophage model, in vivo, in aqueous solutions for oral or parenteral administration, etc.) it is present as penta-O-galloyl-β-D-glucose, whereas in solid form (e.g., in solid unit dosage forms for oral administration), it may be present as the hydrate, which may occur in various degrees of hydration.

Penta-O-galloyl-β-D-glucose hydrate potently reduced the activation of IRAK1, NF-κB, and MAPKs, while increasing expression of IRAK4 in human macrophages through interaction with MyD88. Penta-O-galloyl-β-D-glucose hydrate also inhibited NF-κB translocation into the nucleus. Penta-O-galloyl-β-D-glucose hydrate also suppressed multiple pro-inflammatory cytokines such as IL-1β, TNF-α, IL-6 IL-8, IL-12, MCP-1 and MIP-1α in SARS-CoV-2 Spike (S) protein treated human macrophages, while increasing expression of the anti-inflammatory cytokine IL-10 in nanomolar concentrations, within 2 hours of treatment, without compromising cell viability.

These results indicate that penta-O-galloyl-β-D-glucose enhances type 1 interferon activation, while inhibiting pro-inflammatory cytokine activation and release.

As described herein penta-O-galloyl-β-D-glucose has immune modulating activity in vitro in the presence of SARS-CoV-2, and is expected to have immune modulating activity in the presence of other coronaviruses, such as SARS-CoV, MERS-CoV, common cold coronavirus, as well as influenza viruses, such as influenza A and influenza B. Based on in vitro data, it is possible to enter human clinical trials, as taught in the FDA's Guidance for Industry: Antiviral Product Development—Conducting and Submitting Virology Studies to the Agency (June 2006), which is incorporated herein by reference in its entirety, especially pp. 4-5. See also McMahon et al., “New Approaches for Quantitating the Inhibition of HIV-1 Replication by Antiviral Drugs in vitro and in vivo,” Curr. Opin. Infect. Dis. 2009 December; 22(6): 574-582, which is incorporated herein in its entirety.

Penta-O-galloyl-β-D-glucose hydrate has the formula:

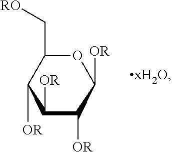

wherein each R is

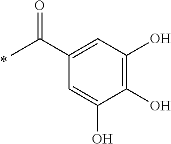

and x is a real number greater than zero.

The immune modulator penta-O-galloyl-β-D-glucose hydrate may be obtained from commercial sources, such as Millipore-Sigma (www.sigmaaldrich.com).

It is expected that compounds of the following Formula XIV will have similar activity to penta-O-galloyl-β-D-glucose, based on their structural similarity to penta-O-galloyl-β-D-glucose, and are thus included within the immune modulatory compounds as described herein.

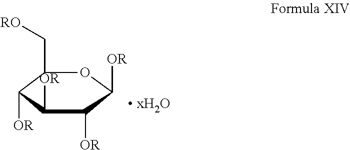

In Formula XIV, x is 0 or a real number greater than zero and each R is independently:

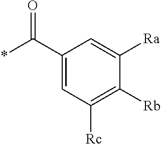

wherein each of Ra, Rb, and Rc is independently a halo (F, Cl, Br, or 1), OH, alkyl, alkoxy, substituted alkyl or alkoxy, wherein the alkyl group of the alkyl or alkoxy may be substituted by one or more halo, OH, or OCH₃.

Compounds of Formula XIV may be synthesized by reacting p-D-glucose with a compound of Formula XV, wherein L is a leaving group, and Ra, Rb and Rc are as defined above, optionally in the presence of a dehydrating agent:

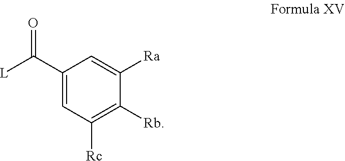

In some embodiments, L may be a halo group (e.g., Cl, Br or 1), an acetyl group (OOC—R′, wherein R′ may be, e.g., methyl, ethyl or isopropyl), as sulfur-based leaving group (such as the tosyl leaving group) or other suitable leaving group.

The effect of penta-O-galloyl-β-D-glucose hydrate on cytokine cytokine response in human macrophages treated with CoV-2 spike protein was tested. Specifically, the effect of penta-O-galloyl-β-D-glucose hydrate on the expression of IL-1α (interleukin 1 alpha), IL-10 (interleukin 10), IFN-γ, VEGF, IL-1B (interleukin 1 beta), IL-2 (interleukin 2), IL-4 (interleukin 4), IL-5 (interleukin 5), IL-6 (interleukin 6), IL-8 (interleukin 8), IL-12p70 (interleukin 12 p70), IL-13 (interleukin 13), GM-CSF (granulocyte-macrophage colony-stimulating factor), GRO (growth related oncogene protein), MCP-1 (monocyte chemoattractant protein-1), MIP-1α (macrophage Inflammatory Protein 1 alpha), MIP-1β (macrophage Inflammatory Protein 1 beta), MMP-9 (matrix metallopeptidase 9), RANTES (Regulated on Activation, Normal T Cell Expressed and Secreted) and TNF-α (tumor necrosis factor alpha), was detected in vitro.

Cinnamanilide

Cinnamanilide (IUPAC name (E)-N,3-diphenylprop-2-enamide) has the chemical structure:

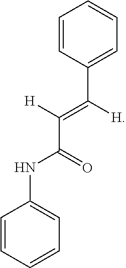

Cinnamanilide has been isolated from the plant species Helichrysum odoratissimum and is commercially available from various sources.

Cinnamanilide inhibited expression of pro-inflammatory cytokines IL-1α, VEGF, IL-6, IL-8, IL-13, GM-CSF, MIP-1α, MIP-1β, MMP-9 and RANTES in macrophages treated with Spike protein. The antiviral compound cinnamanilide activated expression of cytokine modulator IL-10 in macrophages treated with Spike protein. Additionally, cinnamanilide induced apoptosis in macrophages treated with SARS-CoV-2 spike protein by caspase 3 activation.

These results indicate that cinnamanilide enhances type 1 interferon activation, while inhibiting pro-inflammatory cytokine activation and release.

As described herein cinnamanilide has antiviral and immune modulating activity in vitro in the presence of SARS-CoV-2, and is expected to have antiviral and immune modulating activity in the presence of other coronaviruses, such as SARS-CoV, MERS-CoV, common cold coronavirus, as well as influenza viruses, such as influenza A and influenza B. Based on in vitro data, it is possible to enter human clinical trials, as taught in the FDA's Guidance for Industry: Antiviral Product Development—Conducting and Submitting Virology Studies to the Agency (June 2006), which is incorporated herein by reference in its entirety, especially pp. 4-5. See also McMahon et al., “New Approaches for Quantitating the Inhibition of HIV-1 Replication by Antiviral Drugs in vitro and in vivo,” Curr. Opin. Infect. Dis. 2009 December; 22(6): 574-582, which is incorporated herein in its entirety.

It is expected that compounds of the following Formula XVI will have similar antiviral and immune modulating activity to cinnamanilide, based on their structural similarity to cinnamanilide, and are thus included within the antiviral compounds as described herein.

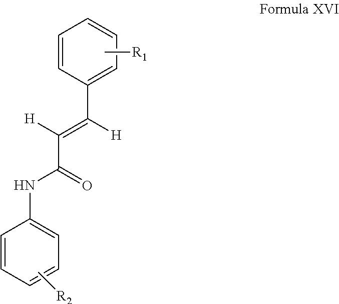

In Formula XVI, each of R₁ and R₂ is H or one to three substituents, independently selected from OH, F, Cl, Br, I, CH₃, CH₂F, CHF₂, or CF₃.

Compounds of Formula I in which R₁ or R₂ is a substituent other than H may be prepared by electrophilic substitution on the phenyl ring by adapting art-recognized methods of electrophilic substitution.

In some preferred embodiments of the antiviral compound of Formula XVI, R₁ may be H, Cl, Br, I, CH₃, or CF₃.

The effect of cinnamanilide on cytokine response in human macrophages treated with CoV-2 spike protein was tested. Specifically, the effect of cinnamanilide on the expression of IL-1α (interleukin 1 alpha), IL-10 (interleukin 10), IFN-γ, VEGF, IL-1β (interleukin 1 beta), IL-2 (interleukin 2), IL-4 (interleukin 4), IL-5 (interleukin 5), IL-6 (interleukin 6), IL-8 (interleukin 8), IL-12p70 (interleukin 12 p70), IL-13 (interleukin 13), GM-CSF (granulocyte-macrophage colony-stimulating factor), GRO (growth related oncogene protein), MCP-1 (monocyte chemoattractant protein-1), MIP-1α (macrophage Inflammatory Protein 1 alpha), MIP-1β (macrophage Inflammatory Protein 1 beta), MMP-9 (matrix metallopeptidase 9), RANTES (Regulated on Activation, Normal T Cell Expressed and Secreted) and TNF-α (tumor necrosis factor alpha), was detected in vitro.

Synthesis of Helichrysetin Derivatives

Helichrysetin derivatives of formula I or formula II in which R₁ to R₄ are alkyl or substituted alkyl may be synthesized by starting helichrysetin as a starting material and substituting for the corresponding H on each corresponding OH group the appropriate R group. One skilled in the art understands how to make these substitutions. For example, one skilled in the art may follow the teaching of U.S. Pat. No. 5,847,170 to Bouchard et al. with respect to substituents R₄ and R₈ disclosed therein, which is incorporated by reference in its entirety, and especially column 7, line 23 through column 11, line 41 and the corresponding examples.

Helichrysetin derivatives of formula I or formula II in which R₈ to R₁₀ are substituents may be prepared by starting with helichrysetin as a starting material and performing suitable ring substitution reactions. One skilled in the art will understand how to carry out such ring substitution reactions.

Synthesis of Cinanserin Derivatives

Cinanserin derivatives of formula III in which R₁ to R₉ are phenyl ring substituents may be prepared by starting with cinanserin as a starting material and performing suitable ring substitution reactions. One skilled in the art will understand how to carry out such ring substitution reactions. Cinanserin derivatives in which one or both R₁₀ is a substituted alkyl may be prepared by a method known in the art.

Baicalin derivatives of formula IV, wherein any one or more of R₁ to R₈ are substituents may be prepared by starting with baicalin as a starting material and performing suitable substitution or acylation reactions. One skilled in the art will understand how to carry out such reactions.

Synthesis of Fangchinoline Derivatives

Fangchinoline derivatives of formula V, wherein any one or more of R₁ to R₁₂ are substituents other than those found naturally in fangchinoline, may be prepared by starting with fangchinoline as a starting material and performing suitable alkyl group or ring substitution reactions. One skilled in the art will understand how to carry out such substitution reactions.

Synthesis of Timosaponin B Derivatives

Timosaponin B derivatives of formula VI R₁ to R₁₃ are substituents may be prepared by starting with timosaponin B as a starting material and performing suitable ring substitution reactions. One skilled in the art will understand how to carry out such ring substitution reactions.

Synthesis of Cepharanthine Derivatives

Cepharanthine derivatives of formula VII, wherein any one or more of R₁ to R₁₁ are substituents, may be prepared by starting with cepharanthine as a starting material and performing suitable ring substitution reactions. One skilled in the art will understand how to carry out such ring substitution reactions.

Synthesis of Tetrandrine Derivatives

Tetrandrine derivatives of formula VIII, wherein any one or more of R₁ to R₁₁ are independently substituents, may be prepared by starting with tetrandrine as a starting material and performing substitution reactions. One skilled in the art will understand how to carry out such substitution reactions.

Synthesis of Bavachalcone B Derivatives

Bavachalcone B derivatives of formula IX, wherein any one or more of R₁ to R₅ are substituents other than those found naturally in bavachalcone B, may be prepared by starting with bavachalcone B as a starting material and performing suitable alkyl group or ring substitution reactions. One skilled in the art will understand how to carry out such substitution reactions.

Synthesis of Rosmarinic Acid Derivatives

Rosmarinic acid derivatives of formula X, wherein any one or more of R₁ to R₇ are substituents other than those found naturally in rosmarinic acid, may be prepared by starting with rosmarinic acid as a starting material and performing suitable acylation or substitution reactions. One skilled in the art will understand how to carry out such acylation or substitution reactions.

Pharmaceutical Compositions

Pharmaceutical compositions described herein comprise helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and one or more additional ingredients and one or more pharmaceutically acceptable ingredients. A “pharmaceutically acceptable” ingredient is an ingredient is compatible with helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and one or more additional ingredients, and with other ingredients of the composition and is suitable for administration to a patient. Additional ingredients may include carriers, diluents, absorption enhancers, stabilizers, preservatives, or other active or inactive ingredients. At least one of the additional ingredients may be an ingredient that does not occur naturally with helichrysetin in nature. In particular, at least one of the additional ingredients may be an ingredient that does not naturally occur with helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, or cinnamanilide in nature. At least one of the additional ingredients may be an ingredient other than water. In some embodiments, the pharmaceutical composition may be sterile, pyrogen free, or both. Sterile, pyrogen free water may be prepared by known means.

In some embodiments, the pharmaceutical composition may be an antiviral composition. The antiviral composition comprises an antivirally effective amount of one or more of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and one or more additional ingredients. The additional ingredient may be an excipient. The excipient may comprise at least one compound that does not occur naturally with helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, or cinnamanilide in nature. In particular, when the antiviral compound is helichrysetin, the excipient may comprise at least one compound that does not naturally occur with helichrysetin in a species of the genus Helichrysum. When the antiviral compound is baicalin, the excipient may comprise at least one compound that does not occur naturally with baicalin in nature. When the antiviral compound is fangchinoline or tetrandrine, the excipient may comprise at least one compound that does not naturally occur with fangchinoline or tetrandrine in Stephania tetrandra. When the antiviral compound is timosaponin B, the excipient may comprise at least one compound that does not occur naturally with timosaponin B in nature. When the antiviral compound is cepharanthine, the excipient may comprise at least one compound that does not naturally occur with cepharanthine in tubers of Stephania. When the antiviral compound is bavachalcone B or rosmarinic acid, the excipient may comprise at least one compound that does not naturally occur with bavachalcone B or rosmarinic acid in a species of the genus Stephania. In some embodiments the excipient may comprise at least one compound other than water. In some embodiments, the additional compound may be a salt or other ingredient at a concentration sufficient for the composition to be isotonic. In some embodiments, the additional ingredient may be a flavor or sweetener not found with helichrysetin in nature. In some embodiments, the antiviral composition may be sterile, pyrogen free, or both.

Pharmaceutical compositions, in particular antiviral compositions, may be formulated for a variety of routes of administration, such as oral, intranasal, intrapulmonary (e.g., for inhalation), intravenous, subcutaneous, transdermal, sublingual, buccal, intraperitoneal, or intrathecal administration. Pharmaceutical compositions may comprise one or more enhancers to assist in the transport of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing against one or more external or internal physiological barriers, such as the blood brain barrier.

Suitable pharmaceutically acceptable excipients may include the following types of excipients: diluents, lubricants, binders, disintegrants, fillers, glidants, granulating agents, coating agents, wetting agents, solvents, co-solvents, suspending agents, emulsifiers, sweeteners, flavoring agents, flavor masking agents, coloring agents, anti-caking agents, humectants, chelating agents, plasticizers, viscosity increasing agents, antioxidants, preservatives, stabilizers, surfactants, and buffering agents.

Antivirally Effective Doses

The effective dose of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may vary depending upon a variety of factors, including the route of administration, the age and condition of the patient in need of antiviral treatment, the species and strain of virus, and the severity of viral infection. In general, helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing are effective in vitro at micromolar concentrations. Effective daily doses of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be in the range of 0.01 mg to 1000 mg per day. The effective daily dose may be divided into two or more divided doses, e.g., 1, 2, 3, 4, 5, 6, or more divided doses. Where the antiviral composition is administered as an infusion, the effective daily dose may be administered as a continuous infusion over a course of hours, e.g., 1-24 hours. An effective dose of an antiviral derivative of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be similar to helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, but may be scaled to account for the greater molecular weight of the derivative compared to parent compound at the compound's relative bioactivity, pharmacokinetics and pharmacodynamics, which one of skill in the art knows how to determine by art-recognized methods.

One skilled in art of pharmaceutical formulation and compounding possesses the knowledge and skill to select suitable pharmaceutically acceptable carriers and excipients in appropriate amounts for the use with helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing. In addition, there are numerous resources available those skilled in the art, which describe pharmaceutically acceptable carriers and excipients and may be useful in selecting suitable pharmaceutically acceptable carriers and excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The compositions of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be prepared using techniques and methods known to those skilled in the art. Some methods commonly used in the art are described in Remington's Pharmaceutical Sciences (Mack Publishing Company).

In some embodiments, antiviral compositions may comprise helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and one or more pharmaceutically acceptable carriers or excipients. The composition may be prepared and packaged in bulk form wherein an effective amount of a compound of the disclosure can be extracted and then given to a subject, such as with powders or syrups. Alternatively, the composition may be prepared and packaged in unit dosage form wherein each physically discrete unit contains an effective amount of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing.

Helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and a pharmaceutically acceptable carrier or excipient(s) will typically be formulated into a dosage form adapted for administration to a subject by a desired route of administration. For example, dosage forms include those adapted for (1) oral administration, such as tablets, capsules, caplets, pills, troches, powders, syrups, elixirs, suspensions, solutions, emulsions, sachets, and cachets; and (2) parenteral administration, such as sterile solutions, suspensions, and powders for reconstitution. Suitable pharmaceutically acceptable carriers or excipients will vary depending upon the particular dosage form chosen. In addition, suitable pharmaceutically acceptable carriers or excipients may be chosen for a particular function that they may serve in the composition. For example, certain pharmaceutically acceptable carriers or excipients may be chosen for their ability to facilitate the production of uniform dosage forms. Certain pharmaceutically acceptable carriers or excipients may be chosen for their ability to facilitate the production of stable dosage forms. Certain pharmaceutically acceptable carriers or excipients may be chosen for their ability to facilitate the carrying or transporting of a compound disclosed herein, once administered to the subject, from one organ or portion of the body to another organ or another portion of the body. Certain pharmaceutically acceptable carriers or excipients may be chosen for their ability to enhance patient compliance.

In some embodiments, antiviral compositions comprising helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, may be formulated for parenteral administration. Compositions adapted for parenteral administration include aqueous and non-aqueous sterile injection solutions which may contain antioxidants, buffers, bacteriostats, and solutes that render the formulation isotonic with the blood of the intended recipient, and aqueous and non-aqueous sterile suspensions that may include suspending agents and thickening agents. The compositions may be presented in unit-dose or multi-dose containers, for example sealed ampoules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets. Parenteral formulations may be sterile, pyrogen-free, or both. Parenteral formulations may be isotonic.

Oral

The antiviral composition may be an oral antiviral compositions comprising helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and at least one excipient suitable for oral administration. The at least one excipient suitable for oral administration may comprise a compound that does not occur naturally with helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing in nature. The at least one excipient suitable for oral administration may comprise at least one compound other than water. Various dosage forms may be prepared, such as tablets, capsules, caplets, troches, powders, emulsions, sachets, cachets, gel capsules, elixirs, pills, oral sprays, chewable tablets, sublingual tablets, films, or sprays, or buccal films or sprays.

In some embodiments, helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be formulated as a solid oral dosage form, such as a tablet or capsule comprising an effective amount of a compound of the disclosure and a diluent or filler. Suitable diluents and fillers include lactose, sucrose, dextrose, mannitol, sorbitol, starch (e.g., corn starch, potato starch, and pre-gelatinized starch), cellulose and its derivatives, (e.g., microcrystalline cellulose), calcium sulfate, and dibasic calcium phosphate. The oral solid dosage form may further comprise a binder. Suitable binders include starch (e.g., corn starch, potato starch, and pre-gelatinized starch) gelatin, acacia, sodium alginate, alginic acid, tragacanth, guar gum, povidone, and cellulose and its derivatives (e.g., microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesium stearate, calcium stearate, and talc.

Where appropriate, dosage unit formulations for oral administration can be microencapsulated. The compositions can also be prepared to prolong or sustain the release as, for example, by coating or embedding particulate material in polymers, wax, or the like.

Helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may also be combined with soluble polymers as targetable drug carriers. Such polymers can include polyvinylpyrrolidone, pyrancopolymer, polyhydroxypropylmethacrylamidephenol, polyhydroxyethylaspartam-idephenol, or polyethylene-oxidepolylysine substituted with palmitoyl residues. Furthermore, helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be combined with a class of biodegradable polymers useful in achieving controlled release of a drug, for example polylactic acid, polepsilon caprolactone, polyhydroxy butyric acid, polyorthoesters, polyacetals, polydihydropyrans, polycyanacrylates and cross-linked or amphipathic block copolymers of hydrogels.

In some embodiments, helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be formulated in a liquid oral dosage form. Oral liquids such as solution, syrups and elixirs can be prepared in dosage unit form so that a given quantity contains a predetermined amount of a compound disclosed herein. Syrups can be prepared by dissolving the compound of the disclosure in a suitably flavored aqueous solution, while elixirs are prepared through the use of a non-toxic alcoholic vehicle. Suspensions can be formulated by dispersing a compound disclosed herein in a non-toxic vehicle. Solubilizers and emulsifiers such as ethoxylated isostearyl alcohols and polyoxy ethylene sorbitol ethers, preservatives, flavor additives such as peppermint oil or other natural sweeteners or saccharin or other artificial sweeteners and the like can also be added.

Intranasal

The antiviral composition may be an intranasal antiviral composition comprising helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and at least one excipient suitable for intranasal administration. The at least one excipient suitable for intranasal administration may comprise at least one compound other than water. For example, the intranasal antiviral composition may comprise one or more penetration enhancers, which increase absorption of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing across the mucosa and/or increase bioavailability. In some embodiments, penetration enhancers may include mucolytic agents, degradative enzyme inhibitors and compounds which increase permeability of the mucosal cell membranes. Whether a given compound is an “enhancer” can be determined by comparing two formulations comprising a non-associated, small polar molecule as the drug, with or without the enhancer, in an in vivo or good model test and determining whether the uptake of the drug is enhanced to a clinically significant degree. The enhancer should not produce any problems in terms of chronic toxicity because in vivo the enhancer should be non-irritant and/or rapidly metabolized to a normal cell constituent that does not have any significant irritant effect. In some embodiments, the penetration enhancer may be an alkyl glycoside, e.g., an alkyl glycoside disclosed in U.S. Pat. No. 5,661,130, which is incorporated herein by reference in its entirety. One skilled in the art recognizes the need to achieve a suitable hydrophile-lipophile balance (HLB) number, which may be determined as disclosed in U.S. Pre-Grant Publication No. US2009/0047347, which is incorporated herein by reference in its entirety.

Intranasal antiviral compositions of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may also include flavors or scents to cover the taste of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing. Intranasal compositions may also include isotonizing agents to make the composition isotonic. Such intranasal antiviral compositions may also include stabilizing agents.

Intrapulmonary

The antiviral composition may be an intrapulmonary antiviral composition comprising helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and at least one excipient suitable for intranasal administration. The at least one excipient suitable for intrapulmonary administration may comprise at least one compound other than water. For example, the intrapulmonary composition may comprise one or more penetration enhancers, which increase the ability of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing to cross the pulmonary epithelia into the blood stream.

Intrapulmonary antiviral compositions may be administered to the lungs by inhalation, e.g., using an insufflator, aerosol inhaler, or a conventional or high efficiency nebulizer.

High efficiency nebulizers are inhalation devices that comprise a microperforated membrane through which a liquid solution is converted through electrical or mechanical means into aerosol droplets suitable for inhalation. High efficiency nebulizers can deliver a large fraction of a loaded dose to a patient. In some embodiments, the high efficiency nebulizer may also utilize one or more actively or passively vibrating microperforated membranes. In some embodiments, the high efficiency nebulizer may comprise one or more oscillating membranes. In some embodiments, the high efficiency nebulizer may comprise a vibrating mesh or plate with multiple apertures and optionally a vibration generator with an aerosol mixing chamber. In some such embodiments, the mixing chamber may function to collect (or stage) the aerosol from the aerosol generator.

In some embodiments, the high efficiency nebulizer may achieve lung deposition (deposited lung dose) of at least about 10% based on the nominal dose of the helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing.

In some embodiments, the high efficiency nebulizer provides lung deposition (deposited lung dose) of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing of at least about 5% based on the nominal dose of the helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing.

In accordance with the invention, in some embodiments, a nebulizer, such as a high efficiency nebulizer may be adapted or adaptable to operate in conjunction with a unit dosage form, such as an ampule or vial, which contains a single dose of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing for antiviral therapy. The unit dosage form comprises a container that contains an inhalation solution comprising the helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing. The container is adapted to cooperate with the high efficiency nebulizer device in such a way as to permit administration of the nominal dose of the inhalation solution to a patient in need thereof. In some embodiments, the high efficiency nebulizer and the unit dosage form are configured so that they are useable together, but not with other devices or dosage forms. In some particular embodiments, the unit dosage form is configured such that it fits into a keyhole-like structure in the high efficiency nebulizer but will not operate with other nebulizer devices. In such embodiments, the high efficiency nebulizer is configured such that it will accept and properly operate with the unit dosage form containing the helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, but not with other dosage forms.

Suitable high efficiency nebulizers with perforated membranes are disclosed in U.S. Pat. Nos. 6,962,151, 5,152,456, 5,261,601, and 5,518,179, each of which is hereby incorporated by reference in its entirety. Suitable high efficiency nebulizers contain oscillatable membranes. Features of these high efficiency nebulizers are disclosed in U.S. Pat. Nos. 7,252,085; 7,059,320; 6,983,747, each of which is hereby incorporated by reference in its entirety.

Commercial high efficiency nebulizers are available from: PARI (Germany) under the trade name eFlow®; Aerogen, Ltd. (Ireland) under the trade names AeroNeb® Go and AeroNeb® Pro, AeroNeb® Solo, and other nebulizers utilizing the OnQ® nebulizer technology; Respironics (Murrysville, Calif.) under the trade names I-Neb©; Omron (Bannockburn, Ill.) under the trade name Micro-Air®; Activaero (Germany) under the trade name Akita®, and AerovectRx (Atlanta, Ga.) under the trade name AerovectRx®.

Conventional nebulizers include, for example jet nebulizers or ultrasonic nebulizers. Jet nebulizers generally utilize compressors to generate compressed air, which breaks the liquid medication into small breathable droplets, which form an aerosolized (atomized) mist. In some of these embodiments, when the patient breathes in, a valve at the top opens, which then allows air into the apparatus, thereby speeding up the mist generation; when the patient breathes out, the top valve closes, thereby slowing down the mist generation while simultaneously permitting the patient to breathe out through the opening of a mouthpiece flap.

Some conventional nebulizers are disclosed in U.S. Pat. Nos. 6,513,727, 6,513,519, 6,176,237, 6,085,741, 6,000,394, 5,957,389, 5,740,966, 5,549,102, 5,461,695, 5,458,136, 5,312,046, 5,309,900, 5,280,784, and 4,496,086, each of which is hereby incorporated by reference in its entirety.

Commercial conventional nebulizers are available from: PARI (Germany) under the trade names PARI LC Plus®, LC Star® and PARI-Jet® A & H Products, Inc. (Tulsa, Okla.) under the trade name AquaTower®; Hudson RCI (Temecula, Calif.) under the trade name AVA-NEB®; Intersurgical, Inc. (Liverpool, N.Y.) underthe trade name Cirrus®; Salter Labs (Arvin, Calif.) under the trade name Salter 8900©; Respironics (Murrysville, Pa.) under the trade name Sidestream®; Bunnell (Salt Lake City, Utah) under the trade name Whisper Jet®; Smiths-Medical (Hyth Kent, UK) under the trade name Downdraft®, and DeVilbiss (Somerset, Pa.) under the trade name DeVilbiss®.

Intravenous

The antiviral composition may be an intravenous antiviral composition comprising helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and at least one excipient suitable for intravenous administration. The at least one excipient suitable for intravenous administration may comprise at least one compound other than water. Intravenous compositions of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing are parenteral compositions intended for intravenous administration by injection or infusion. They may contain one or more isotonizing agents to make the compositions isotonic. They may be, and generally are, sterile, pyrogen free, or both.

Subcutaneous

The antiviral composition may be a subcutaneous antiviral composition comprising helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and at least one excipient suitable for subcutaneous administration. The at least one excipient suitable for subcutaneous administration may comprise at least one compound other than water. Subcutaneous compositions of helichrysetin or a helichrysetin derivative are parenteral compositions intended for injection under the skin. They may contain one or more isotonizing agents to make the compositions isotonic. They may be, and generally are, sterile, pyrogen free, or both.

Transdermal

The antiviral composition may be a transdermal antiviral composition comprising helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing and at least one excipient suitable for transdermal administration. The at least one excipient suitable for transdermal administration may comprise at least one compound other than water. For example, the transdermal antiviral composition may comprise one or more penetration enhancers, which increase the ability of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing to cross the dermis into the blood stream. In addition, the transdermal composition may be delivered by a biasing mechanism, such as an iontophoresis device.

Sublingual or Buccal

The antiviral composition may be a sublingual or buccal antiviral composition comprising helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing and at least one excipient suitable for sublingual or buccal administration. The at least one excipient suitable for sublingual or buccal administration may comprise at least one compound other than water.

Intraperitoneal

The antiviral composition may be an intraperitoneal antiviral composition comprising helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing and at least one excipient suitable for intraperitoneal administration. The at least one excipient suitable for intraperitoneal administration may comprise at least one compound other than water. Intraperitoneal antiviral compositions of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing are parenteral compositions intended for administration to the peritoneum by injection or infusion. They may contain one or more isotonizing agents to make the compositions isotonic. They may be, and generally are, sterile, pyrogen free, or both.

Intrathecal or Intracranioventricular

The antiviral composition may be an intrathecal or intracranioventricular antiviral composition comprising helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and at least one excipient suitable for intrathecal or intracranioventricular administration. The at least one excipient suitable for intrathecal or intracranioventricular administration may comprise at least one compound other than a compound that occurs naturally with helichrysetin in nature, e.g., water. Intrathecal or intracranioventricular antiviral compositions of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing are parenteral compositions intended for administration into the cerebrospinal fluid administration by injection or infusion. They may contain one or more isotonizing agents to make the compositions isotonic. They may be, and generally are, sterile, pyrogen free, or both.

Other Routes of Administration

Although there have been shown and described preferred embodiments of the compositions and methods described herein, it will be readily apparent to those skilled in the art that modifications may be made thereto which do not exceed the scope of the appended claims. Therefore, the scope of the described compositions and methods is only to be limited by the following claims.

Antiviral Methods

Antiviral methods disclosed herein comprise administering an antivirally effective amount of a helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing to a patient in need thereof. A patient in need of an antivirally effective amount of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be a patient having, suspected of having, or being susceptible to acquiring a viral infection. A patient having a viral infection may be a patient who has been diagnosed as having a viral infection, e.g., by a competent medical professional. A patient suspected of having a viral infection may be a patient showing one or more signs or symptoms of a viral infection for whom a diagnosis of viral infection may be tentative or not yet confirmed by definitive testing. A patient susceptible to a viral infection may be any patient whose health, environmental, behavioral, or demographic condition makes the patient vulnerable to infection. A patient may belong to one or more of these categories, and the more categories to which a patient belongs, the more vulnerable the patient may be to infection. A patient whose health makes the patient vulnerable to infection may include patients who are immune compromised, of advanced or very young age, or who have one or more morbidities which make them vulnerable to infection, or if they did acquire an infection, would place them at increased risk of hospitalization, reliance on ventilation or other mechanical life support or life-saving medical intervention. A patient whose environmental or behavioral condition makes the patient more vulnerable to infection may include medical professionals, first responders, and others whose vocation or avocation increases the patient's likelihood of exposure to infection. A patient whose demographic condition makes the patient vulnerable to infection may include patients who are, based on their membership of a particular demographic group, statistically more likely to acquire an infection or to require hospitalization, reliance on ventilation or other mechanical life support or life-saving medical intervention.

Thus, an antivirally effective amount of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may vary depending on the patient's status. In a case in which a patient has a known viral infection, an antivirally effective amount of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be an amount sufficient to reduce the patient's viral load, or to slow an increase in the patient's viral load, or to ameliorate one or more symptoms, or to improve one or more signs of viral infection in the patient. In a case in which a patient is suspected of having a viral infection, an antivirally effective amount of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be an amount sufficient to ameliorate one or more symptoms, or to improve one or more signs of viral infection in the patient. In a case in which a patient is susceptible to acquiring a viral infection, an antivirally effective amount of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be an amount sufficient to reduce the likelihood of the patient acquiring a viral infection or to reduce the severity of a viral infection if one occurs. helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be administered as one of the pharmaceutical compositions disclosed herein. helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be administered as a single therapeutic or in combination with other antiviral, palliative, or supportive therapy. helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be administered to patient having, or suspected of having a viral infection, such as a coronavirus infection or an influenza infection. A coronavirus infection may be an infection of SARS-CoV, MERS-CoV, SARS-CoV2, or a corona virus associated with the common cold. An influenza infection may be caused by Influenza A virus or Influenza B virus.

A patient may be administered a therapeutically effective amount of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing or a prophylactically effective amount of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, which may be administered as a pharmaceutical composition disclosed herein and may be administered as a single therapeutic agent or co-administered with another therapeutic agent. A therapeutically effective amount of helichrysetin or a helichrysetin derivative is an antivirally effective amount of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing effective to treat a patient having, or suspected of having, a viral infection. A prophylactically effective amount of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing is an antivirally effective amount of helichrysetin or a helichrysetin derivative effective to reduce a likelihood of a patient acquiring a viral infection or of reducing the severity of a viral infection.

In light of the disclosure herein, one skilled in the art understand how to determine an antivirally effective amount of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing. Generally, an antivirally effective amount, a therapeutically effective amount, or a prophylactically effective amount, of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be determined, e.g., by inference from in vitro testing. One skilled in the art understands that an effective dose may be inferred from the in vitro half maximal inhibitory concentration (IC₅₀) of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing. For example, in an in vitro inhibition assay against SARS-CoV-2: helichrysetin had an IC₅₀ of 10.43 μM; cinanserin hydrochloride had an IC₅₀ of 13.71 μM; baicalin had an IC₅₀ of 5.96 μM; fangchinoline had an IC₅₀ of 2.05 μM; timosaponin B had an IC₅₀ of 6.11 μM; cepharanthine had an IC₅₀ of 0.51 μM; tetrandrine had an IC₅₀ of 1.25 μM; bavachalcone B had an IC₅₀ of 1.43 μM; and rosmarinic acid had an IC₅₀ of 0.89.

One skilled in the art understands that the effective dose in human patients will depend on the route of administration, the pharmacokinetics, etc. Taking these factors into consideration, an antivirally effective dose of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing may be in the range of 0.1 mg/kg to 150 mg/kg, e.g. 0.1 mg/kg to 1 mg/kg, 0.5 mg/kg to 5 mg/kg, 1 mg/kg to 10 mg/kg, 5 mg/kg to 50 mg/kg, 10 mg/kg to 100 mg/kg, or 50 mg/kg to 150 mg/kg; an effective daily dose of helichrysetin may be some multiple of any of the values within these ranges, e.g. one to six (1 to 6) times the values within these ranges.

Transitional Phrases

In some embodiments, descriptions of the compositions and methods described herein using the transitional word “comprising” indicates that the compositions or methods are “open” to additional ingredients, components or steps. It is intended that “comprising” subsume the more limiting transitional phrases “consisting essentially of” and “consisting of.” Thus, disclosure herein of matter following the transitional phrase “comprising” also fully discloses the same following the transitional phrases “consisting essentially of” or “consisting of.” The transitional phrase “consisting essentially of,” is of intermediate effect, indicating that the subject matter that follows consists only of the recited elements and such additional matter as does not materially affect the novel and basic properties of the claim or claim element. The transitional phrase “consisting of,” indicates that the subject matter that follows is limited to the recited steps or ingredients and is closed to other steps or ingredients not recited. Where a transitional phrase appears within a clause or a sub-clause following another transitional phrase, it is intended that the embedded transitional phrase affect only the phrase in which it appears.

### EXAMPLES

Pharmaceutical compositions and antiviral methods disclosed herein may be further understood with reference to the following illustrative examples.

### Comparative Example 1: In Vitro Antiviral Activity of Comparative Compounds

The in vitro antiviral (SARS-CoV-2) activities of Calpain Inhibitor IV, hydroxychloroquine, chloroquine, E64d (aloxistatin) and remdesivir (comparative compounds) were determined by a cell viability assay. To determine virus inhibition, VERO-E6 cells enriched for angiotensin converting enzyme 2 receptor (ACE-2) were plated into a 384 well titer plate along with SARS-CoV-2 virus. Medium (control) or comparative compound in medium (experimental) at various concentrations was added to wells. Medium-only wells (no cells) were used as controls to determine background luminescence. Cell viability in the presence of SARS-CoV-2 was quantified using the CellTiter-Glo® cell viability assay (Promega, Madison, WI) according to manufacturer's recommendations, converting luminescence values to cell numbers according to a standard curve. Toxicity for each comparative compound was determined by similar methods in the absence of SARS-CoV-2 virus. Table I provides the in vitro activity (IC₅₀) and toxicity (CC₅₀) results for each compound.

TABLE I
In Vitro Anti-SARS-CoV-2 Activity of Positive Controls

| Compound | IC₅₀ (μM) | CC₅₀ (μM) |
|---|---|---|
| Calpain Inhibitor IV | 0.12 | >7.17 |
| Hydroxychloroquine | 3.53 | >30.00 |
| Chloroquine | 3.78 | >30.00 |
| E64d (aloxistatin) | 7.48 | >30.00 |
| Remdesivir | 11.48 | >30.00 |

### Example 1: In Vitro Activity and Toxicity of Antiviral Compounds

The in vitro activity of the antiviral compounds helichrysetin, cinanserin, baicalin, fangchinoline, timosaponin B, cepharanthine, tetrandrine, bavachalcone B, and rosmarinic acid against SARS-Cov-2, the virus that causes COVID-19, was tested in a cell viability assay. To determine virus inhibition, VERO-E6 cells enriched for angiotensin converting enzyme 2 receptor (ACE-2) were plated into a 384 well titer plate along with SARS-CoV-2 virus. Medium (control) or antiviral compound (helichrysetin, cinanserin, baicalin, fangchinoline, timosaponin B, cepharanthine, tetrandrine, bavachalcone B, or rosmarinic acid) in medium (experimental) at various concentrations was added to wells. Medium-only wells (no cells) were used as controls to determine background luminescence. Cell viability in the presence of SARS-CoV-2 was quantified using the CellTiter-Glo® cell viability assay (Promega, Madison, WI) according to manufacturer's recommendations, converting luminescence values to cell numbers according to a standard curve. Table II (1) references the figures containing the graphs showing the activity curves for this assay for SARS-CoV-2 for each of the tabulated antiviral compounds, and (2) summarizes the IC₅₀ values for the antiviral compounds in vitro.

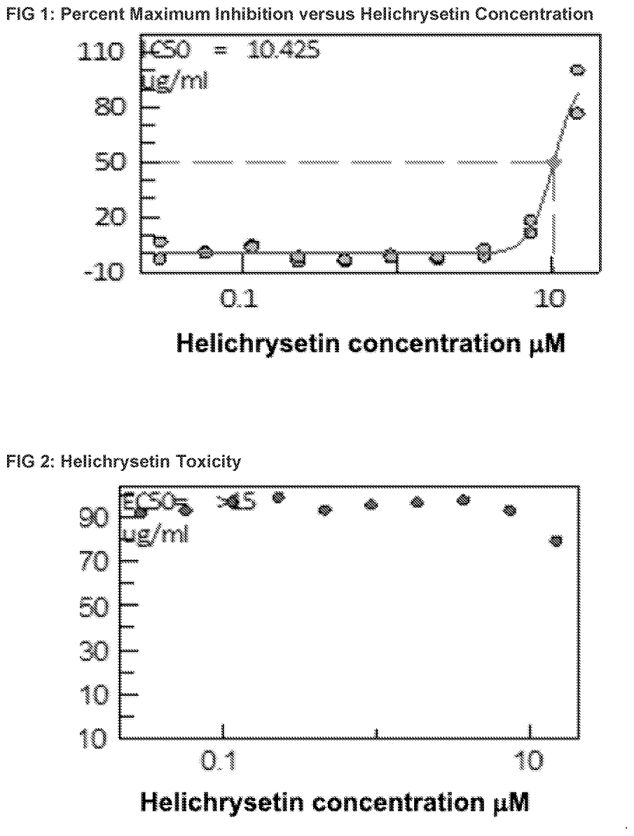

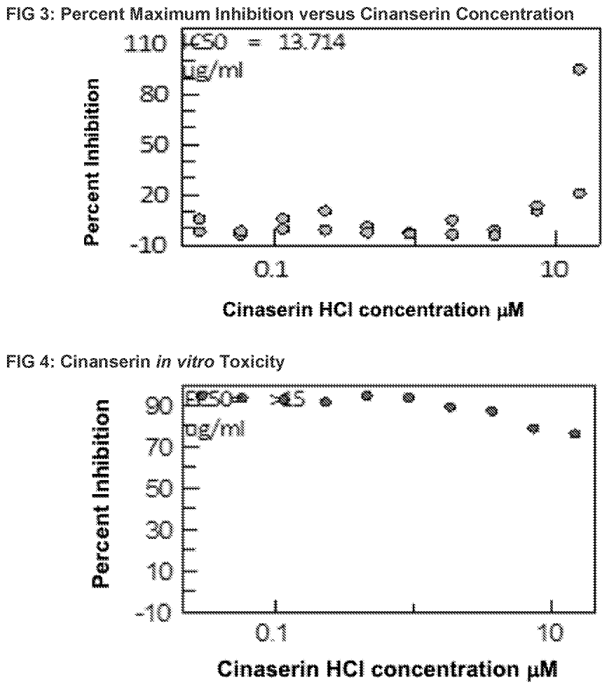

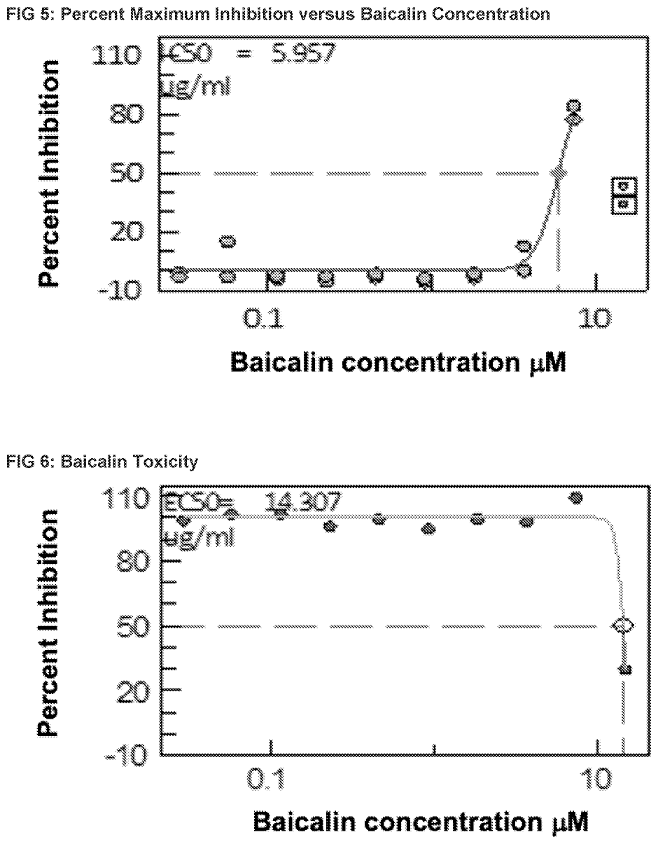

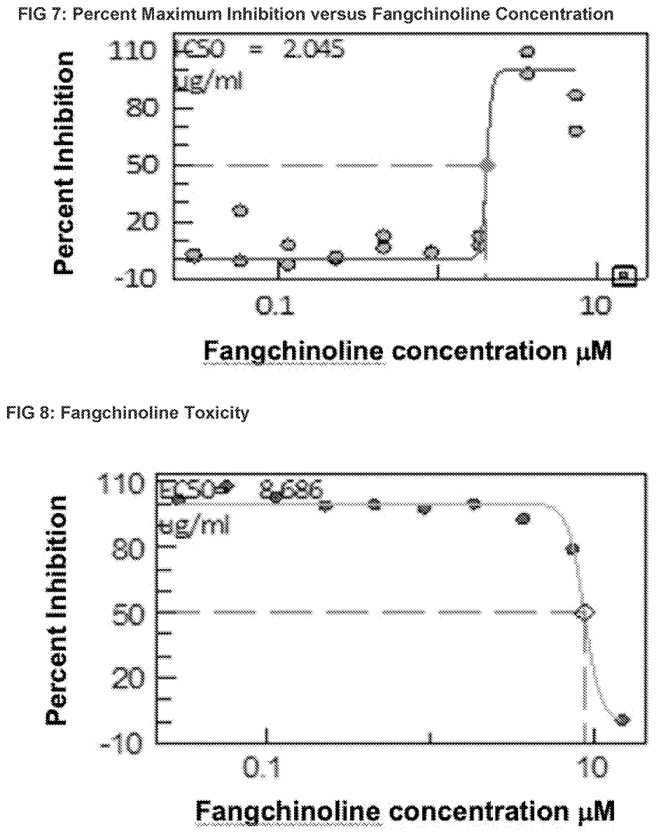

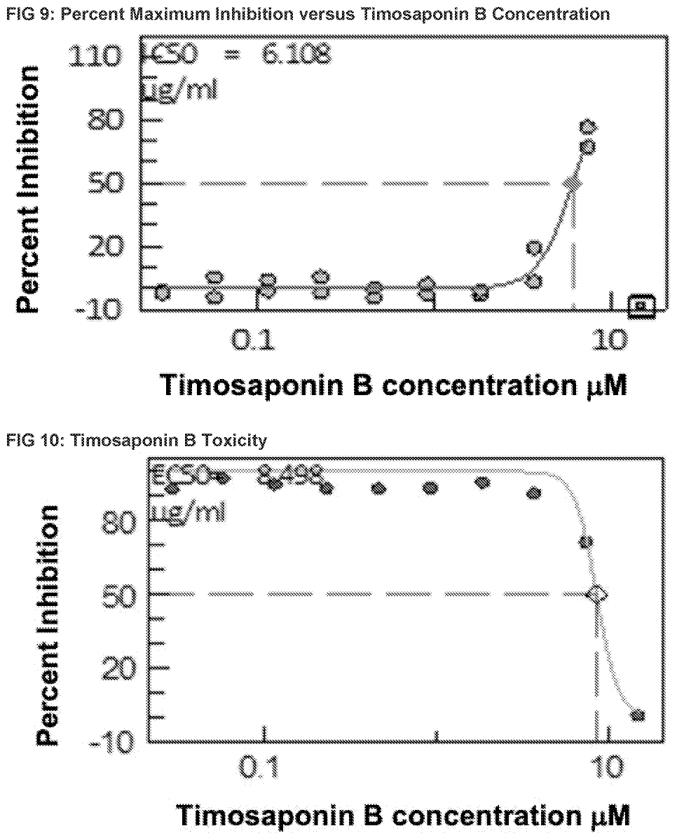

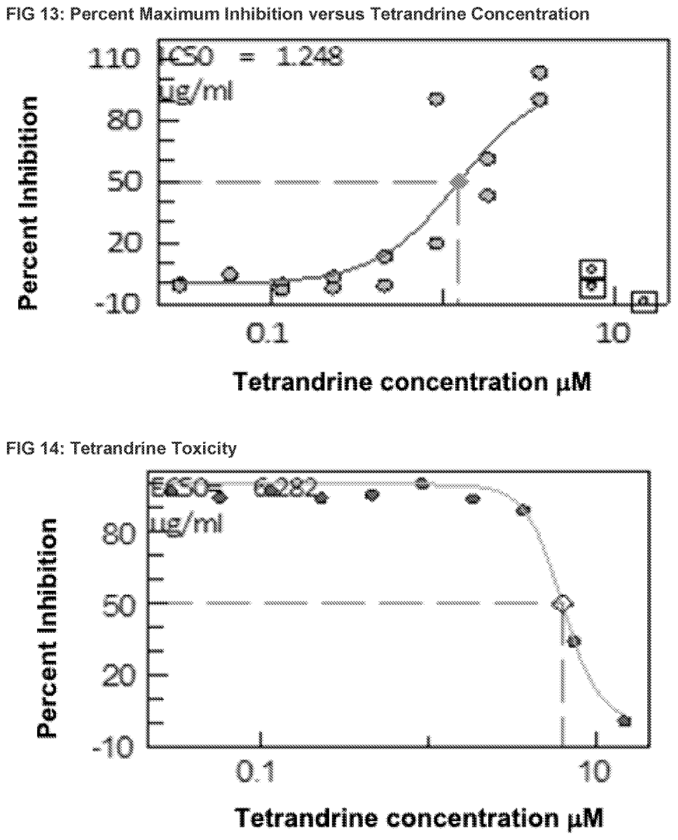

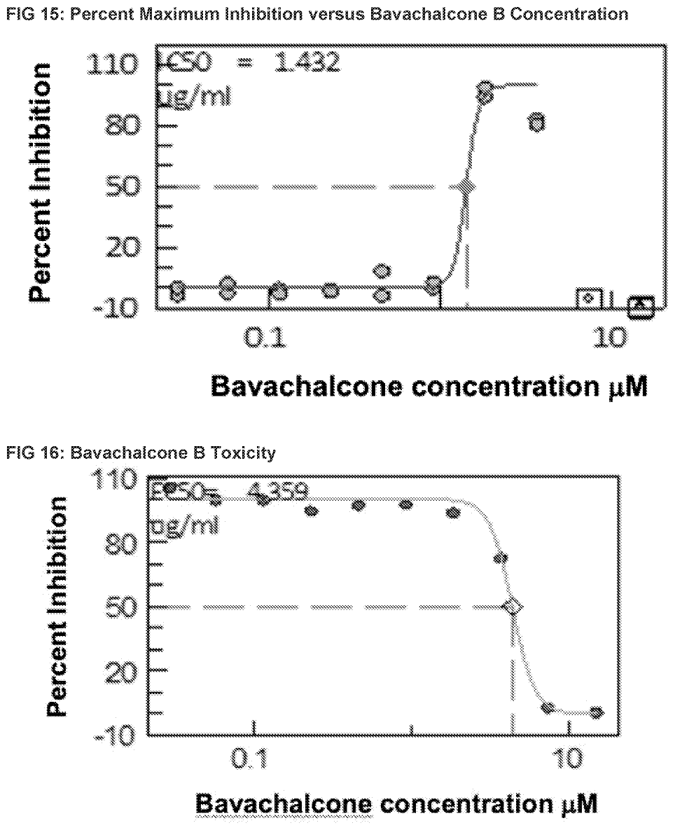

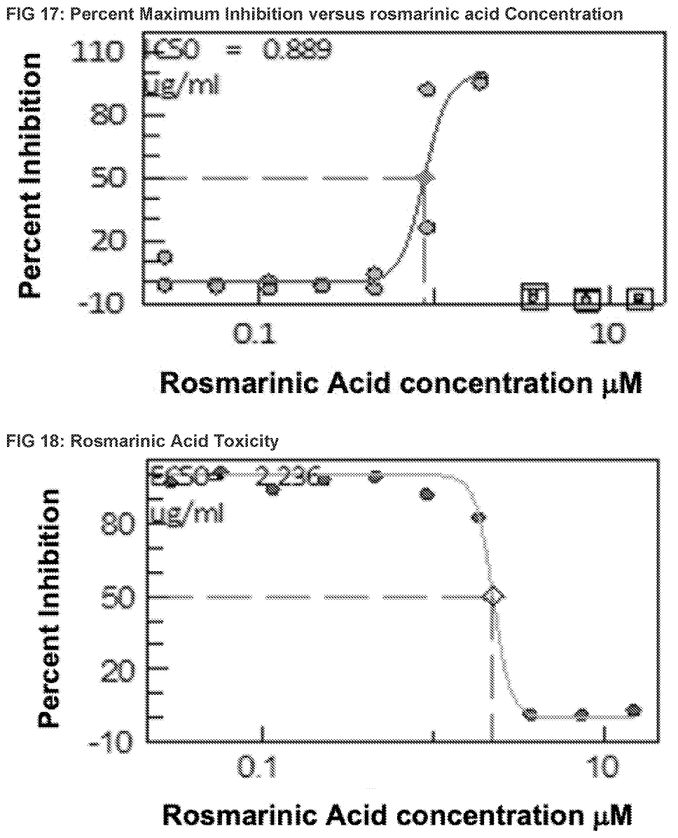

TABLE II
Summary of figures and results for Example 1

| Compound | FIG. No. | IC₅₀ (μM) |
|---|---|---|
| Helichrysetin | FIG. 1 | 10.425 |
| Cinanserin | FIG. 3 | 13.714 |
| Baicalin | FIG. 5 | 5.96 |
| Fangchinoline | FIG. 7 | 2.05 |
| Timosaponin B | FIG. 9 | 6.11 |
| Cepharanthine | FIG. 11 | 0.51 |
| Tetrandrine | FIG. 13 | 1.25 |
| Bavachalcone B | FIG. 15 | 1.43 |
| Rosmarinic acid | FIG. 17 | 0.89 |

To determine toxicity of the antiviral compounds, VERO-E6 cells enriched for angiotensin converting enzyme 2 receptor (ACE-2) were plated into a 384 well titer plate in the absence of virus. Medium (control) or antiviral compound (helichrysetin, cinanserin, baicalin, fangchinoline, timosaponin B, cepharanthine, tetrandrine, bavachalcone B, or rosmarinic acid) in medium (experimental) at various concentrations was added to wells. Medium-only wells (no cells) were used as controls to determine background luminescence. Cell viability in the absence of virus was quantified using the CellTiter-Glo® cell viability assay (Promega, Madison, WI) according to manufacturer's recommendations, converting luminescence values to cell numbers according to a standard curve. The figures showing the in vitro toxicity graphs and the derived CC₅₀ values are set forth in Table Ill.

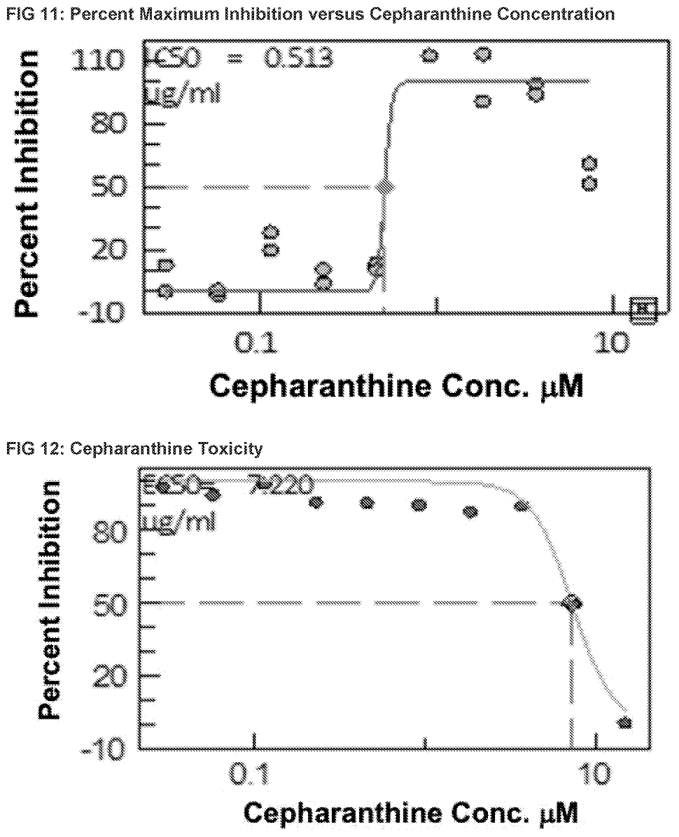

TABLE III
Figures and derived CC₅₀ values for the antiviral compounds

| Compound | FIG. No. | IC₅₀ (μM) |
|---|---|---|
| Helichrysetin | FIG. 2 | >15 |
| Cinanserin | FIG. 4 | >15 |
| Baicalin | FIG. 6 | 14.31 |
| Fangchinoline | FIG. 8 | 8.69 |
| Timosaponin B | FIG. 10 | 8.5 |
| Cepharanthine | FIG. 12 | 7.22 |
| Tetrandrine | FIG. 14 | 6.28 |
| Bavachalcone B | FIG. 16 | 4.36 |
| Rosmarinic acid | FIG. 18 | 2.24 |

As can be seen by comparing the IC₅₀ and CC₅₀ values of the antiviral compounds (helichrysetin, cinanserin, baicalin, fangchinoline, timosaponin B, cepharanthine, tetrandrine, bavachalcone B, or rosmarinic acid) with those of the positive control compounds tested in Comparative Example 1, each of the antiviral compounds (helichrysetin, cinanserin, baicalin, fangchinoline, timosaponin B, cepharanthine, tetrandrine, bavachalcone B, or rosmarinic acid) has favorable in vitro antiviral activity against SARS-CoV-2 and favorable in vitro toxicity.

### Example 2: Activity of Antiviral Compounds Against SARS-CoV and MERS-CoV

The in vitro activities of antiviral compounds (helichrysetin, cinanserin, baicalin, fangchinoline, timosaponin B, cepharanthine, tetrandrine, bavachalcone B, or rosmarinic acid) against SARS-CoV and MERS-CoV were determined by methods analogous to those set forth in Example 1. Each of helichrysetin, cinanserin, baicalin, fangchinoline, timosaponin B, cepharanthine, tetrandrine, bavachalcone B, and rosmarinic acid was found to be active against SARS-CoV and MERS-CoV.

### Example 3: Activity of Antiviral Compounds Against Common Cold Coronaviruses

The in vitro activity of each of the antiviral compounds (helichrysetin, cinanserin, baicalin, fangchinoline, timosaponin B, cepharanthine, tetrandrine, bavachalcone B, or rosmarinic acid) against common cold coronaviruses was determined by methods analogous to those set forth in Example 1. Each of helichrysetin, cinanserin, baicalin, fangchinoline, timosaponin B, cepharanthine, tetrandrine, bavachalcone B, or rosmarinic acid was found to be active against common cold coronavirus.

### Example 4: Activity of the Antiviral Compounds Against Influenza Viruses

The in vitro activity of helichrysetin, cinanserin, baicalin, fangchinoline, timosaponin B, cepharanthine, tetrandrine, bavachalcone B, and rosmarinic acid against Influenza A and Influenza B viruses was determined by methods analogous to those set forth in Example 1. Each of helichrysetin, cinanserin, baicalin, fangchinoline, timosaponin B, cepharanthine, tetrandrine, bavachalcone B, and rosmarinic acid was found to be active against Influenza A and Influenza B.

### Example 5: Antiviral Treatment with an Antiviral Compound or Derivative

An in vitro activity of a derivative of an antiviral compound (helichrysetin, cinanserin, baicalin, fangchinoline, timosaponin B, cepharanthine, tetrandrine, bavachalcone B, or rosmarinic acid) is determined in a manner analogous to that set forth in Example 1.

### Example 6: In Vitro Activity of Antiviral Compounds as Described Herein

The in vitro antiviral and immune modulating effects of fangchinoline, formononetin, baicalein, kazinol A, tetrandrine, cepharanthine, penta-O-beta-glucose hydrate, helichrysetin, and cinnamanilide were tested in the presence of SARS-CoV-2 in lung epithelial cells or macrophages treated with SARS-CoV-2 Spike protein. To determine immune modulation, the methods of Yong, “Cytokine Multiplex Analysis,” Methods Mol. Biol. 2009; 511: 85-105 (July 2019), particularly the PCR and Luminex™ methods, were employed to determine the effect of fangchinoline on expression of cytokines in the macrophages treated with SARS-CoV-2 Spike protein. The Luminex™ method was effected using a Human Cytokine Magnetic 35-Plex Panel (“35-Plex Panel”), from Invitrogen. The 35-Plex Panel can measure expression of 35 cytokines in various sample types: EGF, Eotaxin, FGF basic, G-CSF, GM-CSF, HGF, IFN-α, IFN-γ, IL-1ra, IL-1α, IL-1β, IL-2, IL-2r, IL-3, IL-4, IL-5, IL-6, IL-7, IL-8, IL-9, IL-10, IL-12 (p40/p70), IL-13, IL-15, IL-17A, IL-17F, IL-22, IP-10, MCP-1, MIG, MIP-1α, MIP-1β, RANTES, TNF-α, and VEGF. The results for fangchinoline, formononetin, baicalein, kazinol A, tetrandrine, cepharanthine, penta-O-beta-glucose hydrate, helichrysetin, and cinnamanilide are summarized in Examples 6A-61, below.

### Example 6A: Fangchinoline

The antiviral compound fangchinoline inhibited expression of pro-inflammatory cytokines VEGF, IL-8, MIP-1β and MMP-9 in macrophages treated with Spike protein. Additionally, fangchinoline induced apoptosis in macrophages treated with SARS-CoV-2 spike protein by caspase 3 activation. Based on these results, fangchinoline was identified as a potent modulator of SARS-CoV-2 Spike protein-induced pro-inflammatory cytokines and is expected to have similar activity in vivo.

### Example 6B: Formononetin

The antiviral compound formononetin inhibited expression of pro-inflammatory cytokines VEGF, IL-6, IL-8, GRO, MCP-1, MIP-1α, MIP-1β, MMP-9 and RANTES in macrophages treated with Spike protein. The antiviral compound formononetin activated expression of cytokine modulators IL-10 and IFN-γ in macrophages treated with Spike protein. Additionally, formononetin induced apoptosis in macrophages treated with SARS-CoV-2 spike protein by caspase 3 activation. Based on these results, formononetin was identified as a potent modulator of SARS-CoV-2 Spike protein-induced pro-inflammatory cytokines and is expected to have similar activity in vivo.

### Example 6C: Baicalein

The antiviral compound baicalein inhibited expression of pro-inflammatory cytokines IL-1α, IL-8, GRO, MCP-1, MIP-1α, MIP-1β, MMP-9 and TNF-α in macrophages treated with Spike protein. The antiviral compound baicalein activated expression of cytokine modulators IL-10 and IFN-γ in macrophages treated with Spike protein. Additionally, baicalein induced apoptosis in macrophages treated with SARS-CoV-2 spike protein by caspase 3 activation. Based on these results, baicalein was identified as a potent antiviral compound and modulator of SARS-CoV-2 Spike protein-induced pro-inflammatory cytokines and is expected to have similar activity in vivo.

### Example 6D: Kazinol A

The antiviral compound kazinol A inhibited expression of pro-inflammatory cytokines IL-1α, IL-4, IL-8, MIP-1β and MMP-9 in macrophages treated with Spike protein. The antiviral compound kazinol A activated expression of cytokine modulator IL-10 in macrophages treated with Spike protein. Additionally, kazinol A induced apoptosis in macrophages treated with SARS-CoV-2 spike protein by caspase 3 activation. Based on these results, kazinol A was identified as a potent antiviral compound and modulator of SARS-CoV-2 Spike protein-induced pro-inflammatory cytokines and is expected to have similar activity in vivo.

### Example 6E: Tetrandrine

The antiviral compound, tetrandrine inhibited expression of pro-inflammatory cytokines VEGF, IL-6, IL-8, MIP-1α and MIP-1β, while activating expression of the anti-inflammatory cytokine IL-10, in macrophages treated with Spike protein. Additionally, tetrandrine induced apoptosis in macrophages treated with SARS-CoV-2 spike protein by caspase 3 activation. Based on these results, tetrandrine was identified as a potent antiviral compound and modulator of SARS-CoV-2 Spike protein-induced pro-inflammatory cytokines and is expected to have similar activity in vivo.

### Example 6F: Cepharanthine

The antiviral compound, cepharanthine inhibited expression of pro-inflammatory cytokines VEGF, IL-6, IL-8, GRO, MIP-1α, MIP-1β and MMP-9, while activating expression of the anti-inflammatory cytokine IL-10, in macrophages treated with Spike protein. Additionally, cepharanthine induced apoptosis in macrophages treated with SARS-CoV-2 spike protein by caspase 3 activation. Based on these results, cepharanthine was identified as a potent antiviral compound and modulator of SARS-CoV-2 Spike protein-induced pro-inflammatory cytokines and is expected to have similar activity in vivo.

### Example 6G: Penta-O-Galloyl-β-D-Glucose Hydrate

The immune modulator, penta-O-galloyl-β-D-glucose hydrate inhibited expression of pro-inflammatory cytokines IL-1α, VEGF, IL-1β, IL-2, IL-4, IL-5, IL-6, IL-8, IL-12p70, IL-13, GM-CSF, GRO, MCP-1, MIP-1α, MIP-1β, MMP-9, RANTES, and TNF-α in macrophages treated with Spike protein. The immune modulator penta-O-galloyl-β-D-glucose hydrate activated expression of cytokine modulators IL-10 and IFN-γ in macrophages treated with Spike protein. However, penta-O-galloyl-β-D-glucose hydrate was not observed to induce apoptosis in macrophages treated with SARS-CoV-2 spike protein by caspase 3 activation. Based on these results, penta-O-galloyl-β-D-glucose hydrate was identified as a potent modulator of SARS-CoV-2 Spike protein-induced pro-inflammatory cytokines and is expected to have similar activity in vivo.

### Example 6H: Helichrysetin

The antiviral compound helichrysetin inhibited expression of pro-inflammatory cytokines IL-1α, VEGF, IL-1β, IL-4, IL-5, IL-6, IL-8, IL-12p70, IL-13, MCP-1, MIP-1α, MIP-1β, MMP-9 and TNF-α in macrophages treated with Spike protein. The antiviral compound helichrysetin activated expression of cytokine modulator IL-10 in macrophages treated with Spike protein. However, helichrysetin was not observed to induce apoptosis in macrophages treated with SARS-CoV-2 spike protein by caspase 3 activation. Based on these results, helichrysetin was identified as a potent modulator of SARS-CoV-2 Spike protein-induced pro-inflammatory cytokines and is expected to have similar activity in vivo.

### Example 61: Cinnamanilide

The antiviral compound cinnamanilide activated expression of cytokine modulator IL-10 in macrophages treated with Spike protein. Additionally, cinnamanilide induced apoptosis in macrophages treated with SARS-CoV-2 spike protein by caspase 3 activation. Based on these results, cinnamanilide was identified as a potent modulator of SARS-CoV-2 Spike protein-induced pro-inflammatory cytokines and is expected to have similar activity in vivo.

### Example 7: Antiviral Treatment with an Antiviral Compound or a Derivative

Patients having, or suspected of having, viral infections with SARS-COV, SARS-CoV-2, common cold coronavirus, Influenza A or Influenza B are administered 0.1 mg to 100 mg of helichrysetin, cinanserin, baicalin, fangchinoline, timosaponin B, cepharanthine, tetrandrine, bavachalcone B, or rosmarinic acid one to six times daily. Helichrysetin, cinanserin, baicalin, fangchinoline, timosaponin B, cepharanthine, tetrandrine, bavachalcone B, or rosmarinic acid, or an antiviral derivative, ester, salt, hydrate, anhydrate, polymorph, or tautomer of any of the foregoing, is administered by intranasal, pulmonary, oral, or intravenous route to a patient in need thereof.

### Example 7: Antiviral Prophylaxis with an Antiviral Compound or Derivative

Patients who are susceptible, or suspected of being susceptible, to viral infections with SARS-COV, SARS-CoV-2, common cold coronavirus, Influenza A or Influenza B are administered 0.1 mg to 100 mg of helichrysetin, cinanserin, baicalin, fangchinoline, timosaponin B, cepharanthine, tetrandrine, bavachalcone B, or rosmarinic acid one to six times daily. Helichrysetin, cinanserin, baicalin, fangchinoline, timosaponin B, cepharanthine, tetrandrine, bavachalcone B, or rosmarinic acid, or an antiviral derivative, ester, salt, hydrate, anhydrate, polymorph, or tautomer of any of the foregoing, one to six times daily. helichrysetin, cinanserin, baicalin, fangchinoline, timosaponin B, cepharanthine, tetrandrine, bavachalcone B, or rosmarinic acid one to six times daily. Helichrysetin, cinanserin, baicalin, fangchinoline, timosaponin B, cepharanthine, tetrandrine, bavachalcone B, or rosmarinic acid, or an antiviral derivative, ester, salt, hydrate, anhydrate, polymorph, or tautomer of any of the foregoing, is administered by intranasal, pulmonary, oral, or intravenous route.

While numerous embodiments of pharmaceutical compositions and antiviral methods are described herein, one skilled in the art understand that the examples may be altered to provide other embodiments that utilize the helichrysetin compositions and methods described herein. Therefore, it will be appreciated that the scope of this invention is to be defined by the appended claims rather than by the specific embodiments that have been represented by way of example.

What is claimed is:

1. A unit dose of an antiviral pharmaceutical composition, comprising an antivirally effective amount of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and a pharmaceutically acceptable excipient.

2. The unit dose of claim 1, wherein the pharmaceutically acceptable excipient comprises at least one compound that does not occur naturally with the helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide in nature.

3. The unit dose of claim 1, wherein the helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing is in a chemical form that does not occur in nature.

4. The unit dose of one of claims 1-3, wherein the unit dose is configured for oral administration.

5. The unit dose of claim 4, wherein the unit dose is a tablet, capsule, gel capsule, elixir, pill, oral sprays, chewable tablet, sublingual tablet, film, or spray, or buccal film or spray.

6. The unit dose of one of claims 1-3, wherein the unit dose is configured for intranasal administration.

7. The unit dose of claim 6 in a nasal spray.

8. The unit dose of one of claims 1-3, wherein the unit dose is configured for intrapulmonary administration.

9. The unit dose of claim 8 in a nebulizer.

10. The unit dose of one of claims 1-3, wherein the unit dose is configured for intravenous administration.

11. The unit dose of claim 10 in a sterile solution for intravenous injection.

12. The unit dose of one of claims 1-3, wherein the unit dose is configured for intrathecal or intracerebroventricular administration.

13. The unit dose of claim 12 in a sterile solution for intravenous injection.

14. The unit dose of one of claim 1-3, wherein the unit dose is configured for transdermal administration.

15. The unit dose of claim 14, wherein the pharmaceutically acceptable excipient comprises at least one transdermal penetration enhancer.

16. A dosage container, comprising at least one unit dose of an antiviral pharmaceutical composition comprising an antivirally effective amount of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and a pharmaceutically acceptable excipient.

17. The dosage container of claim 16, wherein the pharmaceutically acceptable excipient comprises at least one compound that does not occur naturally with the helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, or cinnamanilide, in nature.

18. The dosage container of claim 16, wherein the helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide is in a chemical form that does not occur in nature.

19. The dosage container of one of claims 16-18, wherein the unit dose is configured for oral administration.

20. The dosage container of claim 19, wherein the unit dose is a tablet, capsule, gel capsule, elixir, pill, chewable tablet, sublingual tablet, film, or spray, or buccal film or spray.

21. The dosage container of one of claims 16-18, wherein the unit dose is configured for intranasal administration.

22. The dosage container of claim 21 in a nasal spray.

23. The dosage container of one of claims 16-18, wherein the unit dose is configured for intrapulmonary administration.

24. The dosage container of claim 23 in a nebulizer.

25. The dosage container of one of claims 16-18, wherein the unit dose is configured for intravenous administration.

26. The dosage container of claim 25 in a sterile solution for intravenous injection.

27. The dosage container of one of claims 16-18, wherein the unit dose is configured for intrathecal or intracerebroventricular administration.

28. The dosage container of claim 27 in a sterile solution for intravenous injection.

29. The dosage container of one of claim 16-18, wherein the unit dose is configured for transdermal administration.

30. The dosage container of claim 29, wherein the pharmaceutically acceptable excipient comprises at least one transdermal penetration enhancer.

31. An antiviral method of treating a patient, the method comprising administering to the patient in need thereof an antiviral composition comprising an antivirally effective amount of a helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing, and a pharmaceutically acceptable excipient.

32. The method of claim 31, wherein the antiviral composition comprising an antivirally effective amount of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, or an antiviral derivative, ester, salt, hydrate, anhydrate, polymorph, or tautomer of any of the foregoing is in a unit dose.

33. The method of claim 32, wherein the unit dose comprises a pharmaceutically acceptable excipient comprising at least one compound that does not occur naturally with the helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide in nature.

34. The method of claim 32, wherein the helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide is in a chemical form that does not occur in nature.

35. The method of one of claims 31-34, wherein the patient has, is suspected of having, or is susceptible to a coronavirus infection or an influenza virus infection.

36. The method of claim 35, wherein the patient has, is suspected of having, or is susceptible to a coronavirus infection, wherein the coronavirus causing or suspected of causing the infection is a SARS coronavirus, a MERS coronavirus, or a common cold coronavirus.

37. The method of claim 35, wherein the patient has, is suspected of having, or is susceptible to an influenza virus infection, wherein the influenza virus causing or suspected of causing the infection is an influenza A virus or an influenza B virus.

38. The method of one of claims 31-37, wherein the method comprises orally administering the antiviral composition to the patient in need thereof.

39. The method of claim 38, wherein the antiviral composition is a tablet, capsule, gel capsule, elixir, pill, chewable tablet, sublingual tablet, film, or spray, or buccal film or spray.

40. The method of one of claims 31-37, wherein the method comprises intranasally administering the antiviral composition to the patient in need thereof.

41. The method of claim 40, wherein the antiviral composition comprises a nasal spray.

42. The method of one of claims 31-37, wherein the method comprises administering the antiviral composition to the lungs of the patient in need thereof.

43. The method comprising administering the antiviral composition by means of a nebulizer, which may be a high efficiency nebulizer.

44. The method of one of claims 31-37, wherein the method comprises intravenous administration of the antiviral composition to the patient in need thereof.

45. The method of claim 44, wherein the antiviral composition is sterile and pyrogen free.

46. The method of one of claims 31-37, wherein the method comprises intrathecal or intracerebroventricular administration of the antiviral composition to the patient in need thereof.

47. The method of claim 46, wherein the antiviral composition is sterile and pyrogen free.

48. The method of one of claims 32-37, wherein the method comprises transdermal administration of the antiviral composition to the patient in need thereof.

49. The method of claim 48, wherein the pharmaceutically acceptable excipient comprises at least one transdermal penetration enhancer.

50. A compound of formula I: 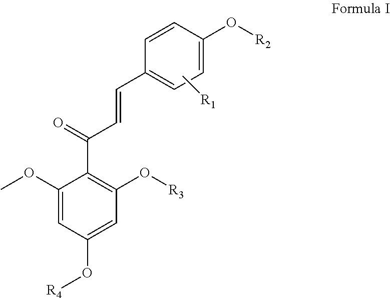
wherein, each of R₁ to R₁₀ is H or a substituent other than H, and at least one of R₁ to R₁₀ is a substituent other than H.

51. The compound of claim 51, wherein the compound is of formula II: 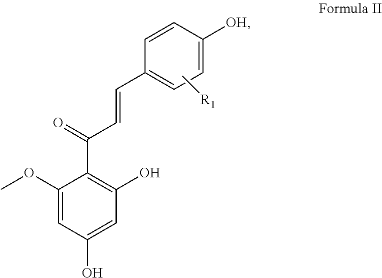
wherein at least one of R₁ to R₄ is other than H.

52. A pharmaceutical composition comprising an antivirally effective amount of a compound of claim 50 or claim 51 and a pharmaceutically acceptable excipient.

53. An antiviral method of treating a patient in need thereof, comprising administering to the patient an antivirally effective amount of a compound of claim 50 or claim 51 or a pharmaceutical composition of claim 52.

54. A compound of formula III: 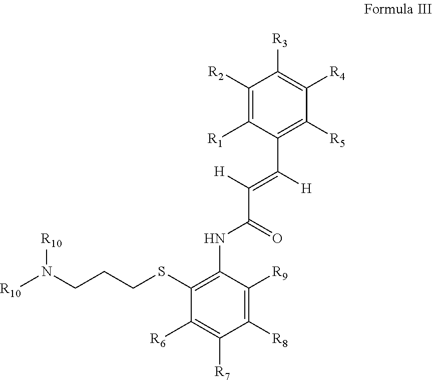
wherein, each of R₁ to R₁₀ is H or a substituent other than H, and at least one of R₁ to R₁₀ is a substituent other than H.

55. A pharmaceutical composition comprising an antivirally effective amount of a compound of claim 54 and a pharmaceutically acceptable excipient.

56. An antiviral method of treating a patient in need thereof, comprising administering to the patient an antivirally effective amount of a compound of claim 55 or a pharmaceutical composition of claim 55.

57. A compound of formula IV: 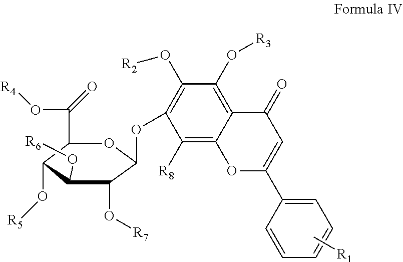
wherein, each of R₁ to R₈ is H or a substituent other than H, and at least one of R₁ to R₈ is a substituent other than H.

58. The compound of claim 57, wherein at least one of R₁ to R₆ is halo or optionally substituted lower alkyl.

59. A pharmaceutical composition comprising an antivirally effective amount of a compound of claim 57 or claim 58 and a pharmaceutically acceptable excipient.

60. An antiviral method of treating a patient in need thereof, comprising administering to the patient an antivirally effective amount of a compound of claim 57 or claim 58 or a pharmaceutical composition of claim 59.

61. A compound of Formula V: 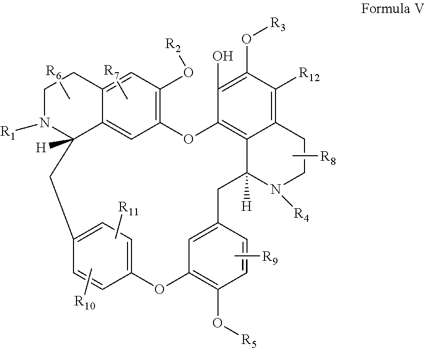
wherein, (1) each of R₆ to R₁₂ is H or a substituent other than H or each of R₁ to R₈ is methyl or a substituent other than methyl; and (2) at least one of R₆ to R₁₂ is a substituent other than H or at least one of R₁ to R₅ is a substituent other than methyl.

62. A pharmaceutical composition comprising an antivirally effective amount of a compound of claim 61 and a pharmaceutically acceptable excipient.

63. An antiviral method of treating a patient in need thereof, comprising administering to the patient an antivirally effective amount of a compound of claim 61 or a pharmaceutical composition of claim 62.

64. A compound of formula VI: 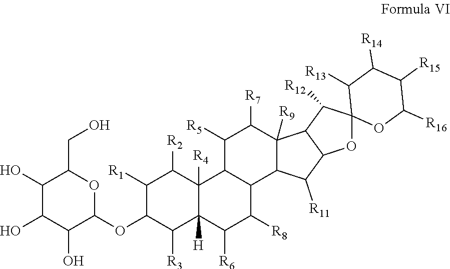
wherein, each of R₁ to R₁₆ is H or a substituent other than H, and at least one of R₁ to R₁₃ is a substituent other than H.

65. A pharmaceutical composition comprising an antivirally effective amount of a compound of claim 64 and a pharmaceutically acceptable excipient.

66. An antiviral method of treating a patient in need thereof, comprising administering to the patient an antivirally effective amount of a compound of claim 65 or a pharmaceutical composition of claim 66.

67. A compound of formula VII: 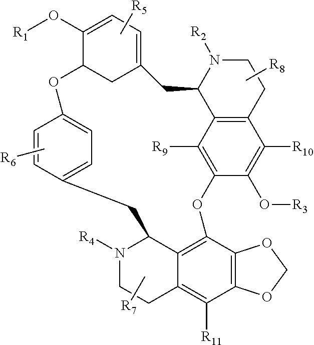
wherein, (1) each of R₅ to R₁₁ is H or a substituent other than H or each of R₁ to R₄ is methyl or a substituent other than methyl; and (2) at least one of R₅ to R₁₁ is a substituent other than H or at least one of R₁ to R₄ is a substituent other than methyl.

68. A pharmaceutical composition comprising an antivirally effective amount of a compound of claim 67 and a pharmaceutically acceptable excipient.

69. An antiviral method of treating a patient in need thereof, comprising administering to the patient an antivirally effective amount of a compound of claim 67 or a pharmaceutical composition of claim 68.

70. A compound of formula VIII: 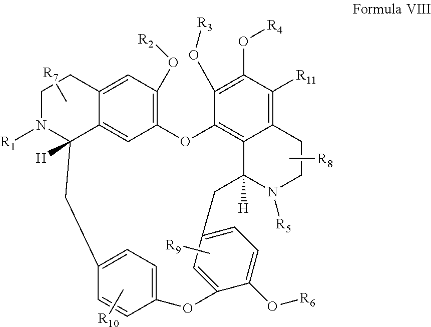
wherein, (1) each of R₇ to R₁₁ is H or a substituent other than H or each of R₁ to R₆ is methyl or a substituent other than methyl; and (2) at least one of R₇ to R₁₁ is a substituent other than H or at least one of R₁ to R₆ is a substituent other than methyl.

71. A pharmaceutical composition comprising an antivirally effective amount of a compound of claim 70 and a pharmaceutically acceptable excipient.

72. An antiviral method of treating a patient in need thereof, comprising administering to the patient an antivirally effective amount of a compound of claim 70 or a pharmaceutical composition of claim 71.

73. A compound of formula IX: 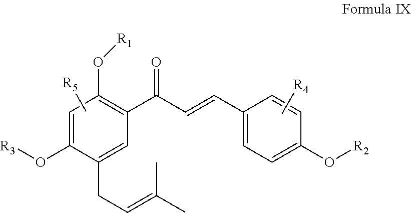
wherein, (1) each of R₄ and R₅ is H or a substituent other than H or each of R₁ to R₃ is methyl or a substituent other than methyl; and (2) at least one of R₄ and R₆ is a substituent other than H or at least one of R₁ to R₃ is a substituent other than methyl.

74. A pharmaceutical composition comprising an antivirally effective amount of a compound of claim 73 and a pharmaceutically acceptable excipient.

75. An antiviral method of treating a patient in need thereof, comprising administering to the patient an antivirally effective amount of a compound of claim 73 or a pharmaceutical composition of claim 74.

76. A compound of formula X: 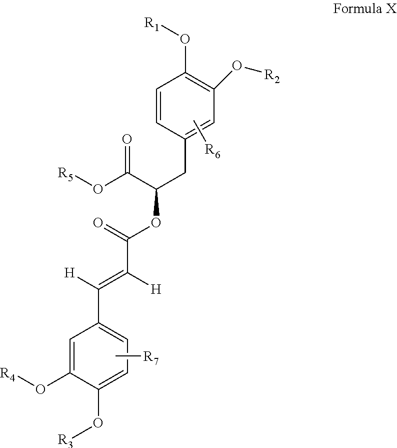
wherein, (1) each of each of R₁ to R₇ is H or a substituent other than H; and (2) at least one of R₁ to R₇ is a substituent other than H.

77. A pharmaceutical composition comprising an antivirally effective amount of a compound of claim 76 and a pharmaceutically acceptable excipient.

78. An antiviral method of treating a patient in need thereof, comprising administering to the patient an antivirally effective amount of a compound of claim 76 or a pharmaceutical composition of claim 77.

79. A compound of Formula XI or Formula XI.A, as defined herein, or a solvate or salt of a compound of Formula XI or Formula XI.A, as defined herein, wherein at least one of R₁ and R₂ is other than H.

80. A pharmaceutical composition comprising a compound of claim 79 and at least one pharmaceutically acceptable excipient.

81. An antiviral method of treating a patient in need thereof, the method comprising administering to the patient in need thereof an antiviral composition comprising a compound of claim 79 or a pharmaceutical composition of claim 80.

82. A unit dose of an antiviral pharmaceutical composition, comprising a pharmaceutically acceptable excipient and an antivirally effective amount of an antiviral compound selected from a compound selected from helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, cinnamanilide, an antiviral derivative, ester, salt, hydrate, anhydrate, polymorph, or tautomer of any of the foregoing, and a compound of any one of claims 50-81.

83. The unit dose of claim 82, wherein the antiviral compound comprises at least one compound selected from the group consisting of helichrysetin, cinanserin, baicalin, fangchinoline, timosaponin B, cepharanthine, tetrandrine, bavachalcone B, rosmarinic acid, formononetin, baicalein, kazinol A, penta-O-beta-glucose hydrate, and cinnamanilide and the pharmaceutically acceptable excipient comprises at least one compound that does not naturally occur with the antiviral compound in nature.
